(19) United States
(12) Patent Application Publication
(10) Pub. No.: US 2002/0164664 A1
(43) Pub. Date: Nov. 7, 2002

(54) Detection and treatment of prostate cancer

(75) Inventors: John J. Hlavaty, Beverly, MA (US); Joseph V. Briggman, Westford, MA (US)

Correspondence Address: TESTA, HURWITZ & THIBEAULT, LLP, HIGH STREET TOWER, 125 HIGH STREET, BOSTON, MA, 02110, US

(73) Assignee: Matritech, Inc.

(21) Appl. No.: 09/998,909

(22) Filed: Nov. 30, 2001

Related U.S. Application Data
(60) Provisional application No. 60/250,284, filed on Nov. 30, 2000.
(60) Provisional application No. 60/344,948, filed on Nov. 8, 2001.

(51) Int. Cl. G01N 33/574

(52) U.S. Cl. 435/7.23

(57) ABSTRACT

The invention provides a wide range of methods and compositions for detecting and treating prostate cancer in an individual. Specifically, the invention provides target prostate cancer-associated proteins, which permit a rapid detection, preferably before metastases occur, of prostate cancer. The target prostate cancer-associated protein may be detected, for example, by reacting the sample with a labeled binding moiety, for example, a labeled antibody capable of binding specifically to the protein. The invention also provides kits useful in the detection of prostate cancer in an individual. In addition, the invention provides methods utilizing the prostate cancer-associated proteins either as targets for treating prostate cancer or as indicators for monitoring the efficacy of such a treatment.

### REFERENCE TO RELATED APPLICATION

This application claims priority to U.S. Serial No. 60/250,284, filed Nov. 30, 2000, and to U.S. Serial No. 60/______ , “Materials and Methods for Detection and Treatment of Prostate Cancer,” Attorney Docket No. MTP-027PR2, filed Nov. 8, 2001, the disclosures of each of which are incorporated by reference herein.

### BACKGROUND OF THE INVENTION

Among American men, prostate cancer is the most frequently occurring cancer and the second-leading cause of cancer-related deaths. Death usually results not from the primary lesion but from secondary lesions, for example, metastases to lymph nodes or bone marrow. Significant efforts are underway worldwide not only to develop tools to improve the detection of prostate cancer but also to determine the aggressiveness or metastatic potential of the cancer.

Prostate specific antigen (PSA) is widely used as a diagnostic tool for prostate cancer (see U.S. Pat. No. 5,501,983). Although serum PSA levels are elevated in men with prostate cancer, they are elevated also in men with non-malignant disorders including prostatitis and benign prostatic hyperplasia (BPH). Although most serum PSA is present in a complex with alpha-1-antichymotrypsin, some is present in an uncomplexed (free) state. Generally, a higher proportion of PSA is free in patients with BPH than in patients with prostate cancer, permitting some improvement in the discrimination between these two conditions. Nevertheless, PSA levels do not indicate the aggressiveness of a prostate cancer. Because the progression of prostate cancers is highly variable, the inability of PSA to assess the metastatic potential or state of a cancer underlines the need for the development of other prostate cancer markers.

Prostatic acid phosphatase (PAP) serum assays are available as a diagnostic tool for prostate cancer. Unfortunately, PAP assays suffer from a variety of drawbacks including intraday fluctuations in serum PAP levels and contamination with acid phosphatases from other tissues. Serum PAP assays are therefore inferior to PSA assays for most purposes (see, Lowe et al., (1993) in The Urologic Clinics of North America: Prostatic Tumor Markers, Oesterling, J. E., ed., 20(4):589-595).

In recent years, a wide variety of other prostate cancer markers have been proposed, although none has gained widespread acceptance. For example, prostate stem cell antigen mRNA is overexpressed in most prostate cancers, although its expression has not been correlated to tumor stage or grade (see, Reiter et al., (1998) Proc. Natl. Acad. Sci. USA, 95:1735-1740). Changes in the methylation state of GSTP 1 have been observed in prostate cancers, but the susceptibility of the assay to false negative and false positive results may limit its acceptance in clinical settings (see, Lee et al., (1997) Cancer Epidemiology, Biomarkers & Prevention, 6(6):387-474). Prostate cancer-1 (PC-1) protein is present at elevated levels in the nuclear matrix fraction of prostate carcinoma samples when compared to normal prostate samples (see, U.S. Pat. No. 5,824,490). In addition, serum levels of a marker known as MPS-N correlate with the presence of prostate cancer. MPS-N levels, however, do not appear to be a better predictor of cancer than PSA levels are (see, Fernandez-Pol et al., (1997) Anticancer Res., 17:1519-1530).

Some markers may help to distinguish between prostate cancer and benign hypertrophy. RT-PCR assays detect prostate carcinoma tumor antigen-1 (PCTA-1) more often in samples from prostatic carcinoma than in samples from normal prostate or from BPII (see, Su et al., (1996) Proc. Natl. Acad. Sci. USA, 93:7252-7). Similarly, analysis of serum levels of glandular kallikrein 2, in conjunction with PSA assays, improves discrimination between BPH and prostate cancer (see, Becker et al., (2000) J. Urology, 163:311-316).

Other markers have been proposed as prognostic indicators. High mobility group protein-1 (Y) mRNA expression correlates with the stage and grade of prostate tumors, although the assay used for its detection, RNA in situ hybridization, is difficult and does not lend itself to clinical applications (see, Tamimi et al., (1996) Br. J. Cancer, 74:573-8). Similarly, although the levels of YL-1 in nuclear matrix fractions from prostate tissues correlate with the presence of aggressive cancer in the tissues when analyzed by two-dimensional gel electrophoresis, these assays are laborious and expensive (see, Lakshmanan et al., (1998) J. Urology, 159:1354-1358). Aggressive prostate cancers have also been reported to have higher levels of type IV collagenase (see, Stearns et al., (1993) Cancer Research, 53:878-883). RT-PCR assays suggest that the alternative splicing of fibroblast growth receptor 2 may be different in androgen-dependent and—independent forms of prostate cancer (see, Carstens et al., (1997) Oncogene, 15:3059-3065).

Despite the recent flurry of candidate markers for prostate cancer detection and prognosis, there remains a significant, long-felt need for (i) markers with improved sensitivity and specificity, (ii) markers that can better distinguish between hyperplasia and malignancy, (iii) markers that can accurately predict the course of the disease, and (iv) markers that can discriminate between aggressive and nonaggressive cancers. There remains a need for assays that are inexpensive, simple, rapid, and useful within and without clinical settings.

### SUMMARY OF THE INVENTION

The invention provides methods and compositions for detecting the presence of prostate cancer in a mammal, for example, a human, and for treating prostate cancer in a mammal diagnosed with the disease. The invention is based, in part, upon the discovery of a family of proteins each member of which is detectable at a higher concentration in serum from a mammal, for example, a human, with prostate cancer relative to serum from a normal mammal, that is, a mammal without prostate cancer. Accordingly, these proteins, as well as nucleic acid sequences encoding such proteins, or sequences complementary thereto, can be used as prostate cancer markers useful in diagnosing prostate cancer, monitoring the efficacy of a prostate cancer therapy, and/or as targets of such a therapy. Indeed, at least one of these markers permits detection of more than ninety percent of all prostate cancers, including prostate cancers that are undetectable by PSA assays.

In one aspect, the invention provides a novel protein species that has been identified as a highly effective marker for prostate cancer. The protein species has been shown to include a polypeptide that is related to Vitamin D binding protein (VDBP). The polypeptide includes amino acid sequences that are also found in VDBP. Accordingly, the protein species of the invention may be characterized as including a polypeptide having at least one sequence selected from the group consisting of SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO:10, and SEQ ID NO:11, any of which are believed to distinguish VDBP-related proteins from other proteins. Alternatively, a protein species of the invention may be characterized as including a polypeptide having both the sequence of SEQ ID NO:4 and SEQ ID NO:12, which in combination are believed to distinguish VDBP-related proteins from other proteins, or may be characterized as binding specifically to an anti-VDBP antibody.

The protein species is distinguishable from previously known protein species by its biochemical characteristics and/or by its sequence. For example, in a solution of 50 mM sodium phosphate, pH 7.0, the protein species may bind to an anion exchange resin bearing quarternary ammonium groups. Following washes of the resin with solutions of 25 mM sodium chloride, 50 mM sodium chloride, and 75 mM sodium chloride, each in 50 mM sodium phosphate, pH 7.0, the protein species elutes from the resin in the presence of a solution of 125 mM sodium chloride in 50 mM sodium phosphate, pH 7.0. The protein species preferably has a mass of about 50.8 kD as measured by matrix-activated laser desorption and ionization-time of flight (MALDI-TOF) mass spectrometry using a derivatized chip surface, and preferably has an affinity for a derivatized chip surface bearing carboxyl groups, or for a derivatized chip surface bearing nickel ions. As used herein, “mass spectrometry” encompasses the terms “mass spectrometry” and “mass spectroscopy.”

In another aspect, the invention provides methods for diagnosing cancer by detecting, in a sample from an individual, a polypeptide indicative of the presence of the cancer and including either a sequence selected from the group consisting of SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO:10, and SEQ ID NO:11, or including both SEQ ID NO:4 and SEQ ID NO:12. The method preferably detects a polypeptide that is present in samples from more than fifty percent of males with prostate cancer and absent in males from more than fifty percent of males without prostate cancer. The polypeptide may bind to an anion exchange resin in 50 mM sodium phosphate, pH 7.0, and elute from the resin in 125 mM sodium chloride in 50 mM sodium phosphate, pH 7.0, as described above. Absences of the polypeptide in samples from males with prostate cancer are “false negatives,” whereas presences of the polypeptide in samples from males without prostate cancer are “false positives.” Preferably, the false negative and false positive rates are each less than 40%, and more preferably less than 30%, more preferably less than 20%, more preferably less than 10%, and most preferably less than or equal to 5%. The sample may be or include tissue, for example, prostate tissue, or a body fluid such as serum, plasma, prostatic secretion, blood, sweat, tears, urine, peritoneal fluid, lymph, semen, seminal fluid, seminal plasma, spinal fluid, ascitic fluid, saliva, or sputum. Preferred methods include measuring the concentration of the polypeptide in the sample and comparing the concentration of the polypeptide with a threshold value indicative of the presence of prostate cancer.

The invention also provides methods of diagnosing prostate cancer by contacting a sample from an individual with a binding moiety that binds specifically to a cancer-associated protein to produce a complex containing the binding moiety and the cancer-associated protein, and detecting the complex, which if present is indicative of prostate cancer in the individual. The cancer-associated protein includes a polypeptide having at least one sequence selected from the group consisting of SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO:10, and SEQ ID NO:11, and/or having both the sequence of SEQ ID NO:4 and the sequence of SEQ ID NO:12. The polypeptide may optionally bind to an anion exchange resin in 50 mM sodium phosphate, pH 7.0, and elute from the resin in 125 mM sodium chloride in 50 mM sodium phosphate, pH 7.0, as described above. The binding moiety may be an antibody, such as a monoclonal or polyclonal antibody, and is preferably labeled with a detectable moiety, such as a radioactive label, a hapten label, a fluorescent label, a chemiluminescent label, a spin label, a colored label, and an enzymatic label.

The invention also provides other cancer-associated protein markers. The protein markers are characterized as being detectable at a higher concentration in the serum of a mammal, specifically, a human, with cancer than in serum of a mammal without cancer. The protein markers can be detectable at a higher concentration in the serum of a mammal with disseminated prostate cancer than in the serum of a mammal with localized (e.g. organ-confined) prostate cancer. Alternatively, the protein markers can be detectable at a higher concentration in the serum of a mammal with an elevated prostate-specific antigen (PSA) level and prostate cancer than in the serum of a mammal with an elevated prostate-specific antigen (PSA) level but without prostate cancer.

One marker protein is further characterized in that it has a molecular weight of about 21 kD, binds to an anion exchange resin in the presence of 50 mM sodium phosphate, pH 7.0, and elutes from the anion exchange resin in the presence of 125 mM sodium chloride in 50 mM sodium phosphate, pH 7.0. This marker protein also has a binding affinity to an H4 SELDI chip.

Another marker protein is further characterized in that it has a molecular weight of about 23 kD, binds to an anion exchange resin in the presence of 50 mM sodium phosphate, pH 7.0, and elutes from the anion exchange resin in the presence of 175 mM sodium chloride in 50 mM sodium phosphate, pH 7.0. This marker protein also has a binding affinity to a nickel SELDI chip.

Another marker protein is further characterized in that it has a molecular weight of about 25 kD, binds to an anion exchange resin in the presence of 50 mM sodium phosphate, pH 7.0, and elutes from the anion exchange resin in the presence of 125 mM sodium chloride in 50 mM sodium phosphate, pH 7.0. This marker protein also has a binding affinity to a WCX-2 SELDI chip.

Another marker protein is further characterized in that it has a molecular weight of about 26 kD, binds to an anion exchange resin in the presence of 50 mM sodium phosphate, pH 7.0, and elutes from the anion exchange resin in the presence of 100 mM sodium chloride in 50 mM sodium phosphate, pH 7.0. This marker protein also has a binding affinity to an H4 SELDI chip.

Another marker protein is further characterized in that it has a molecular weight of about 51 kD, binds to an anion exchange resin in the presence of 50 mM sodium phosphate, pH 7.0, and elutes from the anion exchange resin in the presence of 125 mM sodium chloride in 50 mM sodium phosphate, pH 7.0. This marker protein also has a binding affinity to a nickel SELDI chip.

Another marker protein is further characterized in that it has a molecular weight of about 60 kD, and binds poorly to an anion exchange resin in the presence of 50 mM sodium phosphate, pH 7.0. This marker protein also has a binding affinity to a nickel SELDI chip.

Another marker protein is further characterized in that it has a molecular weight of about 125 kD, binds to an anion exchange resin in the presence of 50 mM sodium phosphate, pH 7.0, and elutes from the anion exchange resin in the presence of 125 mM sodium chloride in 50 mM sodium phosphate, pH 7.0. This marker protein also has a binding affinity to a nickel SELDI chip.

Furthermore, the aforementioned prostate cancer-associated proteins are further characterized as being non-immunoglobulin and/or non-albumin proteins. Furthermore, the prostate cancer-associated proteins may further define an antigenic region or epitope that may bind specifically to a binding moiety, for example, an antibody, for example, a monoclonal or a polyclonal antibody, an antibody fragment thereof, or a biosynthetic antibody binding site directed against the antigenic region or epitope. In addition, the invention enables one skilled in the art to isolate nucleic acids encoding the aforementioned prostate cancer-associated proteins or nucleic acids capable of hybridizing under specific hybridization conditions to a nucleic acid encoding the prostate cancer-associated proteins. Furthermore, the skilled artisan may produce nucleic acid sequences encoding the entire isolated marker protein, or fragments thereof, using methods currently available in the art (see, for example, Sambrook et al., eds. (1989) Molecular Cloning: A Laboratory Manual, Cold Spring Harbor Press). For example, the prostate cancer-associated protein of the invention, when isolated, can be sequenced using conventional peptide sequencing protocols. Based on the peptide sequence, it is possible to produce oligonucleotide hybridization probes useful in screening a cDNA library. The cDNA library may then be screened with the resultant oligonucleotide to isolate full or partial length cDNA sequences encoding the isolated protein.

Each of these markers may be used, alone or in combination, in methods such as protein or nucleic acid-based methods for detecting the presence of cancer in a mammal. The methods of the invention may be performed on any relevant tissue or body fluid sample. For example, methods of the invention may be performed on prostate tissue, more preferably prostate biopsy tissue. Alternatively, the methods of the invention may be performed on a body fluid sample selected from the group consisting of: blood; serun; plasma; fecal matter; urine; semen, seminal fluid, seminal plasma, prostatic secretion; spinal fluid; saliva; ascitic fluid; peritoneal fluid; and sputum. It is contemplated, however, that the methods of the invention also may be useful in detecting metastasized prostate cancer cells in other tissue or body fluid samples.

Detection of prostate cancer can be accomplished using any one of a number of assay methods well known and used in the art. In one aspect, the method of diagnosing cancer in an individual comprises contacting a sample from the individual with a first binding moiety that binds specifically to a prostate-cancer associated protein to produce a first binding moiety-cancer-associated protein complex. The first binding moiety is capable of binding specifically to at least one of the prostate cancer associated marker proteins identified hereinabove to produce a complex. Thereafter the presence and/or amount of marker protein in the complex can then be detected, for example, via the first binding moiety if labeled with a detectable moiety, for example, a radioactive or fluorescent label, or a second binding moiety labeled with a detectable moiety that binds specifically to the first binding moiety using conventional methodologies well known in the art. The presence or amount of the marker protein can thus be indicative of the presence of prostate cancer in the individual. For example, the amount of marker protein in the sample may be compared against a threshold value previously calibrated to indicate the presence or absence of prostate cancer, wherein the amount of the complex in the sample relative to the threshold value can be indicative of the presence or absence of cancer in the individual. Although such a method can be performed on tissue, for example, prostate tissue, or a body fluid, for example, serum, a body fluid currently is the preferred test sample.

Detection of the aforementioned nucleic acid molecules can also serve as an indicator of the presence of prostate cancer and/or metastasized prostate cancer in an individual. Accordingly, in another aspect, the invention provides another method for detecting prostate cancer in a human. The method comprises the step of detecting the presence of a nucleic acid molecule in a tissue or body fluid sample thereby to indicate the presence of prostate cancer in an individual. The nucleic acid molecule is selected from the group consisting of (i) a nucleic acid molecule comprising a sequence capable of recognizing and being specifically bound by a prostate cancer-associated protein, and (ii) a nucleic acid molecule comprising a sequence encoding at least a portion of one or more of the prostate cancer-associated proteins identified herein.

In one embodiment, the method comprises exposing a sample from the individual under specific hybridization conditions to a nucleic acid probe, for example, greater than about 10 and more preferably greater than 15 nucleotides in length, capable of hybridizing to a target nucleic acid encoding one of the prostate cancer-associated proteins identified herein to produce a duplex. Thereafter, the presence of the duplex can be detected using a variety of detection methods known and used in the art. It is contemplated that the target nucleic acid may be amplified, for example, via conventional polymerase chain reaction (PCR) or reverse transcriptase polymerase chain reaction (RT-PCR) methodologies, prior to hybridization with the nucleic acid probe.

In one embodiment, the target nucleic acid (for example, a messenger RNA (mRNA) molecule), is greater than 15 nucleotides, more preferably greater than 50 nucleotides, and most preferably greater than 100 nucleotides in length and encodes an amino acid sequence present in one of the prostate cancer-associated proteins identified herein. Such a target mRNA may then be detected, for example, by Northern blot analysis by reacting the sample with a labeled hybridization probe, for example, a ³²P labeled oligonucleotide probe, capable of hybridizing specifically with at least a portion of the nucleic acid molecule encoding the marker protein. Detection of a nucleic acid molecule either encoding a prostate cancer-associated protein or capable of being specifically bound by a prostate cancer-associated protein, can thus serve as an indicator of the presence of a prostate cancer in the individual being tested.

In another aspect, the invention provides a kit for detecting the presence of prostate cancer or for evaluating the efficacy of a therapeutic treatment of a prostate cancer. Such kits may comprise, in combination, (i) a receptacle for receiving a human tissue or body fluid sample from the individual to be tested, (ii) a binding partner which binds specifically either to an epitope on a prostate cancer-associated marker protein or a nucleic acid sequence encoding at least a portion of the prostate cancer-associated protein or the nucleic acid sequence encoding at least a portion of the prostate cancer-associated protein, and (iii) a reference sample. In one embodiment, the reference sample may comprise a negative and/or positive control. In that embodiment, the negative control would be indicative of a normal prostate cell type and the positive control would be indicative of prostate cancer. Such a kit may also be used for identifying potential candidate therapeutic agents for treating cancer.

In another aspect, the invention provides methods and compositions for treating prostate cancer. In one aspect the invention provides proteins or nucleobase-containing sequences useful in the treatment of prostate cancer. The therapeutic protein could be, for example, a binding moiety, for example, an antibody, for example, a monoclonal antibody, an antigenic binding fragment thereof, or a biosynthetic antibody binding site capable of binding specifically to a prostate cancer-associated protein identified herein. The method comprises the step of administering to a patient with prostate cancer, a therapeutically-effective amount of a compound, preferably an antibody, and most preferably a monoclonal antibody, which binds specifically to a target prostate cancer-associated protein thereby to inactivate or reduce the biological activity of the protein. The target protein may be any of the prostate cancer-associated proteins identified herein. Similarly, it is contemplated that the compound may comprise a small molecule, for example, a small organic molecule, which inhibits or reduces the biological activity of the target prostate cancer-associated protein.

In another aspect, the invention provides another method for treating prostate cancer. The method comprises the step of administering to a patient diagnosed as having prostate cancer, a therapeutically-effective amount of a compound which reduces in vivo the expression of a target prostate cancer-associated protein thereby to reduce in vivo the expression of the target protein. In a preferred embodiment, the compound is a nucleobase containing sequence, for example, an anti-sense nucleic acid sequence or a peptidyl nucleic acid (PNA) capable of binding to and reducing the expression (for example, transcription or translation) of a nucleic acid encoding at least a portion of at least one of the prostate cancer-associated proteins identified herein. After administration, the anti-sense nucleic acid sequence or the anti-sense PNA molecule binds to the nucleic acid sequences encoding, at least in part, the target protein thereby to reduce in vivo expression of the target prostate cancer-associated protein.

Thus, the invention provides a wide range of methods and compositions for detecting and treating prostate cancer in an individual. Specifically, the invention provides prostate cancer-associated proteins, which permit specific and early, preferably before metastases occur, detection of prostate cancer in an individual. In addition, the invention provides kits useful in the detection of prostate cancer in an individual. In addition, the invention provides methods utilizing the prostate cancer-associated proteins as targets and indicators, for treating prostate cancers and for monitoring of the efficacy of such a treatment. These and other numerous additional aspects and advantages of the invention will become apparent upon consideration of the following figures, detailed description, and claims.

### BRIEF DESCRIPTION OF THE DRAWINGS

FIG. 1 is a schematic representation of an apparatus used to electroelute a representative marker protein from an acrylamide gel slice.

FIG. 2 shows the nucleotide (SEQ ID NO:13) and amino acid (SEQ ID NO:14) sequences of an allele of Vitamin D binding protein (VDBP). Fragments of the protein corresponding to protein fragments identified as cancer markers are underlined.

FIG. 3 shows a Western blot analysis of 125 mM sodium chloride HPLC fractions of serum samples from males with and without prostate cancer. The Western blot is probed with a polyclonal antibody to VDBP.

FIGS. 4A-4F shows mass spectroscopic analyses of serum fractions from males with prostate cancer. FIG. 4A shows the profile of a fraction after repeated exposure to an anti-fetuin antibody; FIG. 4B shows protein retained by the anti-fetuin antibody. FIG. 4C shows the profile of a fraction after repeated exposure to an anti-α-1-antitrypsin antibody; FIG. 4D shows protein retained by the anti-α-1-antitrypsin antibody. FIG. 4E shows the profile of a fraction after repeated exposure to an anti-VDBP antibody; and FIG. 4F shows protein retained by the antibody.

### DETAILED DESCRIPTION OF THE INVENTION

The present invention provides methods and compositions for the detection and treatment of prostate cancer. The invention is based, in part, upon the discovery of prostate cancer-associated proteins which generally are present at detectably higher levels in serum of humans with prostate cancer relative to serum of humans without prostate cancer.

The prostate cancer-associated proteins or nucleic acids encoding such proteins may act as markers useful in the detection of prostate cancer or as targets for therapy of prostate cancer. For example, it is contemplated that the marker proteins and binding moieties, for example, antibodies that bind to the marker proteins or nucleic acid probes which hybridize to nucleic acid sequences encoding the marker proteins, may be used to detect the presence of prostate cancer in an individual. Furthermore, it is contemplated that the skilled artisan may produce novel therapeutics for treating prostate cancer which include, for example: antibodies which can be administered to an individual that bind to and reduce or eliminate the biological activity of the target protein in vivo; nucleic acid or peptidyl nucleic acid sequences which hybridize with genes or gene transcripts encoding the target proteins, thereby to reduce expression of the target proteins in vivo; or small molecules, for example, organic molecules which interact with the target proteins or other cellular moieties, for example, receptors for the target proteins, thereby to reduce or eliminate biological activity of the target proteins.

Set forth below are methods for isolating prostate cancer-associated proteins, methods for detecting prostate cancer using prostate cancer-associated proteins as markers, and methods for treating individuals afflicted with prostate cancer using prostate cancer-associated proteins as targets for cancer therapy.

Methods for Detecting Prostate Cancer-Associated Marker Proteins.

Marker proteins of the invention, as disclosed herein, are identified by comparing the protein composition of serum of a human diagnosed with prostate cancer with the protein composition of serum of a human free of prostate cancer. As used herein, the term “prostate cancer-associated protein” is understood to mean any protein which is detectable at a higher level in a tissue or body fluid of an individual diagnosed with prostate cancer relative to a corresponding tissue or body fluid of an individual free of prostate cancer and includes species and allelic variants thereof and fragments thereof. As used herein, the term “prostate cancer” is understood to mean any cancer or cancerous lesion associated with prostate tissue or prostate tissue cells and can include precursors to prostate cancer. It is not necessary that the marker protein or target molecule be unique to a prostate cancer cell or body fluid of an individual afflicted with prostate cancer; rather the marker protein or target molecule should have a signal to noise ratio high enough to discriminate between samples originating from a prostate cancer tissue or body fluid and samples originating from normal prostate tissue or body fluid.

As used herein, a “portion” or a “fragment” of a protein or of an amino acid sequence denotes a contiguous peptide comprising, in sequence, at least ten amino acids from the protein or amino acid sequence (e.g. amino acids 1-10, 34-43, or 127-136 of the protein or sequence). Preferably, the peptide comprises, in sequence, at least twenty amino acids from the protein or amino acid sequence. More preferably, the peptide comprises, in sequence, at least forty amino acids from the protein or amino acid sequence.

As used herein with reference to anion exchange chromatography, a protein in a particular solution “weakly binds” a chromatography column if at least ten percent of the protein is present in the column flow-through upon adequate washing of the column with that solution.

The prostate cancer-associated marker proteins of the invention were identified by comparing the proteins present in the serum of individuals with prostate cancer to the proteins present in the serum of individuals without prostate cancer. Albumin and immunoglobulin proteins were removed from the serum, and the proteins were separated into fourteen fractions by anion exchange chromatography. Briefly, the proteins were loaded on a strong anion exchange column in the presence of 50 mM sodium phosphate, pH 7.0, and eluted with a stepwise gradient of sodium chloride in 50 mM sodium phosphate, pH 7.0. The resulting fourteen fractions include a flow-through fraction, a fraction eluting in 25 mM sodium chloride, a 50 mM fraction, a 75 mM fraction, a 100 mM fraction, a 125 mM fraction, a 150 mM fraction, a 175 mM fraction, a 200 mM fraction, a 225 mM fraction, a 250 mM fraction, a 300 mM fraction, a 400 mM fraction, and a 2 M fraction.

Each fraction was analyzed by (surface-enhanced laser desorption and ionization (SELDI)) mass spectrometry. Samples from each of the twelve fractions were applied to one of four different SELDI chip surfaces. A nickel SELDI surface can be generated by adding a nickel salt solution to a chip comprising ethylenediaminetriacetic acid. Other SELDI chip surfaces include: WCX-2 which comprises carboxylate moieties, SAX-2 which comprises quarternary ammonium moieties, and H4 which is hydrophobic. The prostate cancer-associated proteins of the invention can therefore be characterized by their increased presence in serum of individuals having prostate cancer relative to individuals without prostate cancer, their molecular weight, binding and elution characteristics on an anion exchange resin, and their affinity to a particular SELDI chip. For example, as used herein, the term “affinity” to a particular SELDI chip is understood to mean that the prostate cancer-associated proteins of the invention bind preferentially to one type of SELDI chip (e.g., H4 SELDI chip) relative to one or more of the other SELDI chips (e.g., the nickel, SAX-2 and WCX-2 chips) disclosed herein. As discussed in detail in Example 1, comparison of the sera from diseased and healthy individuals revealed a number of proteins frequently present at detectable levels in the sera of diseased individuals, but infrequently present at comparable levels in the sera of healthy individuals.

Once the prostate cancer-associated proteins have been identified by mass spectroscopy, the identified proteins can be isolated by standard protein isolation methodologies and sequenced using protein sequencing technologies known and used in the art. Once the amino acid sequences are identified then nucleic acids encoding the marker proteins or portions thereof can be identified by comparing the sequences to a database of known or predicted translation products of known nucleic acid sequences, such as those identified through the Human Genome Project. Appropriate databases are available, for example, through the National Center for Biotechnology Information.

Marker proteins useful in the present invention encompass not only the particular sequences identified herein but also allelic variants thereof and related proteins that also function as marker proteins. Thus, for example, sequences that result from alternative splice forms, post-translational modification, or gene duplication are each encompassed by the present invention. Species variants are also encompassed by this invention where the patient is a non-human mammal. Other homologous proteins that may function as marker proteins are also envisioned.

Preferably, variant sequences are at least 80% similar or 70% identical, more preferably at least 90% similar or 80% identical, and most preferably 95% similar or 90% identical to at least a portion of one of the sequences disclosed herein.

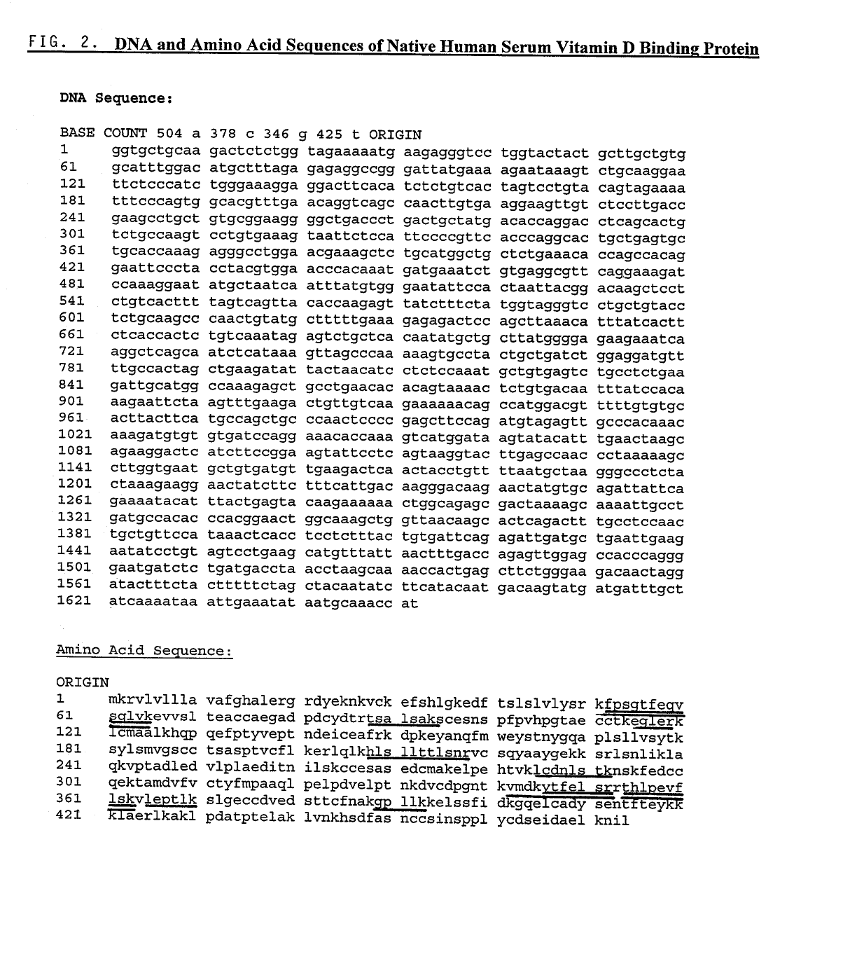

To determine whether a candidate peptide region has the requisite percentage similarity or identity to a reference polypeptide or peptide oligomer, the candidate amino acid sequence and the reference amino acid sequence are first aligned using the dynamic programming algorithm described in Smith and Waterman (1981), J. Mol. Biol. 147:195-197, in combination with the BLOSUM62 substitution matrix described in FIG. 2 of Henikoff and Henikoff (1992), PNAS 89:10915-10919. For the present invention, an appropriate value for the gap insertion penalty is −12, and an appropriate value for the gap extension penalty is −4. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and reference sequence is made, a percent similarity score may be calculated. The individual amino acids of each sequence are compared sequentially according to their similarity to each other. If the value in the BLOSUM62 matrix corresponding to the two aligned amino acids is zero or a negative number, the pairwise similarity score is zero; otherwise the pairwise similarity score is 1.0. The raw similarity score is the sum of the pairwise similarity scores of the aligned amino acids. The raw score is then normalized by dividing it by the number of amino acids in the smaller of the candidate or reference sequences. The normalized raw score is the percent similarity. Alternatively, to calculate a percent identity, the aligned amino acids of each sequence are again compared sequentially. If the amino acids are non-identical, the pairwise identity score is zero; otherwise the pairwise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity. Accordingly, gap penalties are not used in this calculation, although they are used in the initial alignment.

In all instances, variants of the naturally-occurring sequences, as described above, must be tested for their function as marker proteins. Specifically, their presence or absence in a particular form or in a particular biological compartment must be indicative of the presence or absence of cancer in an individual. This routine experimentation can be carried out by the methods described hereinbelow or by other methods known in the art.

Marker proteins in a sample of tissue or body fluid may be detected via binding assays, wherein a binding partner for the marker protein is introduced into a sample suspected of containing the marker protein. In such an assay, the binding partner may be detectably labeled as, for example, with a radioisotopic or fluorescent marker. Labeled antibodies may be used in a similar manner in order to isolate selected marker proteins. Nucleic acids encoding marker proteins may be detected using nucleic acid probes having a sequence complementary to at least a portion of the sequence encoding the marker protein. Techniques such as PCR and, in particular, reverse transcriptase PCR, are useful means for isolating nucleic acids encoding a marker protein. The examples which follow provide details of the isolation and characterization of prostate cancer-associated proteins and methods for their use in the detection and treatment of prostate cancer.

Detection of Prostate Cancer

Once prostate cancer-associated proteins have been identified, the proteins or nucleic acids encoding the proteins may be used as markers to determine whether an individual has prostate cancer and, if so, suitable detection methods can be used to monitor the status of the disease.

Using the marker proteins or nucleic acids encoding the proteins, the skilled artisan can produce a variety of detection methods for detecting prostate cancer in a human. The methods typically comprise the steps of detecting, by some means, the presence of one or more prostate cancer-associated proteins or nucleic acids encoding such proteins in a tissue or body fluid sample of the human. The accuracy and/or reliability of the method for detecting prostate cancer in a human may be further enhanced by detecting the presence of a plurality of prostate cancer-associated proteins and/or nucleic acids in a preselected tissue or body fluid sample. The detection assays may comprise one or more of the protocols described hereinbelow.

2.A. Protein-Based Assays

The marker protein in a sample may be detected using a variety of techniques known in the art, a subset of which are discussed below.

In one approach, for example, the marker protein may be detected using a binding moiety capable of specifically binding the marker protein. The binding moiety may comprise, for example, a member of a ligand-receptor pair, i.e., a pair of molecules capable of having a specific binding interaction. The binding moiety may comprise, for example, a member of a specific binding pair, such as antibody-antigen, enzyme-substrate, nucleic acid-nucleic acid, protein-nucleic acid, protein-protein, or other specific binding pair known in the art. Binding proteins may be designed which have enhanced affinity for a target protein. Optionally, the binding moiety may be linked with a detectable label, such as an enzymatic, fluorescent, radioactive, phosphorescent or colored particle label. The labeled complex may be detected, e.g., visually or with the aid of a spectrophotometer or other detector.

A prostate cancer-associated marker protein may be detected using any of a wide range of immunoassay techniques available in the art. For example, the skilled artisan may employ the sandwich immunoassay format to detect prostate cancer in a body fluid sample. Alternatively, the skilled artisan may use conventional immuno-histochemical procedures for detecting the presence of the prostate cancer-associated protein in a tissue sample using one or more labeled binding proteins.

In a sandwich immunoassay, two antibodies capable of binding the marker protein generally are used, e.g., one immobilized onto a solid support, and one free in solution and labeled with a detectable chemical compound. Examples of chemical labels that may be used for the second antibody include radioisotopes, fluorescent compounds, spin labels, colored particles such as colloidal gold and colored latex, and enzymes or other molecules that generate colored or electrochemically active products when exposed to a reactant or enzyme substrate. When a sample containing the marker protein is placed in this system, the marker protein binds to both the immobilized antibody and the labeled antibody, to form a “sandwich” immune complex on the support's surface. The complexed protein is detected by washing away non-bound sample components and excess labeled antibody, and measuring the amount of labeled antibody complexed to protein on the support's surface. Alternatively, the antibody free in solution, which can be labeled with a chemical moiety, for example, a hapten, may be detected by a third antibody labeled with a detectable moiety which binds the free antibody or, for example, the hapten coupled thereto.

Both the sandwich immunoassay and tissue immunohistochemical procedures are highly specific and very sensitive, provided that labels with good limits of detection are used. A detailed review of immunological assay design, theory and protocols can be found in numerous texts in the art, including Butt, W. R., ed. (1984) Practical Immunology Marcel Dekker, N. Y. and Harlow et al. eds. (1988) Antibodies, A Laboratory Approach, Cold Spring Harbor Laboratory.

In general, immunoassay design considerations include preparation of antibodies (e.g., monoclonal or polyclonal antibodies) having sufficiently high binding specificity for the target protein to form a complex that can be distinguished reliably from products of nonspecific interactions. As used herein, the term “antibody” is understood to mean binding proteins, for example, antibodies or other proteins comprising an immunoglobulin variable region-like binding domain, having the appropriate binding affinities and specificities for the target protein. The higher the antibody binding specificity, the lower the target protein concentration that can be detected. As used herein, the terms “specific binding” or “binding specifically” are understood to mean that the binding moiety, for example, a binding protein has a binding affinity for the target protein of greater than about 10⁵ M⁻¹, more preferably greater than about 10⁷ M⁻¹.

Antibodies to an isolated target prostate cancer-associated protein which are useful in assays for detecting a prostate cancer in an individual may be generated using standard immunological procedures well known and described in the art. See, for example, Practical Immunology, Butt, N. R., ed., Marcel Dekker, NY, 1984. Briefly, an isolated target protein is used to raise antibodies in a xenogeneic host, such as a mouse, goat or other suitable mammal. The marker protein is combined with a suitable adjuvant capable of enhancing antibody production in the host, and is injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (e.g., cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt (1984) (supra).

In addition, genetically engineered biosynthetic antibody binding sites, also known in the art as BABS or sFv's, may be used in the practice of the instant invention. Methods for making and using BABS comprising (i) non-covalently associated or disulfide bonded synthetic V_{H} and V_{L} dimers, (ii) covalently linked V_{H}-V_{L} single chain binding sites, (iii) individual V_{H} or V_{L} domains, or (iv) single chain antibody binding sites are disclosed, for example, in U.S. Pat. Nos.: 5,091,513; 5,132,405; 4,704,692; and 4,946,778. Furthermore, BABS having requisite specificity for the prostate cancer-associated proteins can be derived by phage antibody cloning from combinatorial gene libraries (see, for example, Clackson et al. (1991) Nature 352: 624-628; or U.S. Pat. No. 5,837,500). Briefly, phage each expressing on their coat surfaces BABS having immunoglobulin variable regions encoded by variable region gene sequences derived from mice pre-immunized with isolated prostate cancer-associated proteins, or fragments thereof, are screened for binding activity against immobilized prostate cancer-associated protein. Phage which bind to the immobilized prostate cancer-associated proteins are harvested and the gene encoding the BABS is sequenced. The resulting nucleic acid sequences encoding the BABS of interest then may be expressed in conventional expression systems to produce the BABS protein.

Marker proteins may also be detected using gel electrophoresis techniques available in the art. In two-dimensional gel electrophoresis, the proteins are separated first in a pH gradient gel according to their isoelectric point. The resulting gel then is placed on a second polyacrylamide gel, and the proteins separated according to molecular weight (see, for example, O'Farrell (1975) J. Biol. Chem. 250: 4007-4021; or Berkelman et al. (Oct. 1998) “2-D Electrophoresis Using Immobilized pH Gradients: Principles and Methods,” Amersham Pharmacia Biotech Pub. 80-6429-60, Rev. A).

One or more marker proteins may be detected by first isolating proteins from a sample obtained from an individual suspected of having prostate cancer, and then separating the proteins by two-dimensional gel electrophoresis to produce a characteristic two-dimensional gel electrophoresis pattern. The pattern may then be compared with a standard gel pattern produced by separating, under the same or similar conditions, proteins isolated from normal or cancer cells. The standard gel pattern may be stored in, and retrieved from an electronic database of electrophoresis patterns. The presence of a prostate cancer-associated protein in the two-dimensional gel provides an indication that the sample being tested was taken from a person with prostate cancer. As with the other detection assays described herein, the detection of two or more proteins, for example, in the two-dimensional gel electrophoresis pattern further enhances the accuracy of the assay. The presence of a plurality, e.g., two to five, prostate cancer-associated proteins on the two-dimensional gel provides an even stronger indication of the presence of a prostate cancer in the individual. The assay thus permits the early detection and treatment of prostate cancer.

Mass spectrometry may also be used to detect a marker protein. Preferred mass spectrometry methods include MALDI-TOF mass spectrometry and MALDI-TOF using derivatized chip surfaces (SELDI). Useful mass spectrometry methods for detecting a marker protein are described, for example, in the Examples and in U.S. Pat. Nos. 5,719,060; 6,124,137; 6,207,370; 6,225,047; 6,281,493; and 6,322,970.

These detection methods may be used in combination with each other, with other detection methods, and/or with one or more purification methods to reduce the complexity of a biological sample. Thus, for example, proteins isolated by two-dimensional gel electrophoresis could be probed with an antibody that specifically binds the marker protein, or could be assayed by mass spectrometry. Similarly, as described in the Examples, a biological sample may be subjected to biochemical fractionation prior to analysis by mass spectrometry or by other techniques such as gel electrophoresis and/or immunoassays. A marker protein may also be detected indirectly, for example, by subjecting it to enzymatic treatment, and subsequently detecting the products of that treatment, as demonstrated in Example 3, in which a marker protein was digested with an endopeptidase (trypsin), and the resulting fragments were detected by liquid chromatography-mass spectrometry (LC-MS).

The isolated prostate cancer-associated protein also may be used for the development of diagnostic and other tissue evaluating kits and assays to monitor the level of the proteins in a tissue or fluid sample. For example, the kit may include antibodies or other specific binding proteins which bind specifically to the prostate cancer-associated proteins and which permit the presence and/or concentration of the prostate cancer-associated proteins to be detected and/or quantitated in a tissue or fluid sample.

Suitable kits for detecting prostate cancer-associated proteins are contemplated to include, e.g., a receptacle or other means for capturing a sample to be evaluated, and means for detecting the presence and/or quantity in the sample of one or more of the prostate cancer-associated proteins described herein. As used herein, “means for detecting” in one embodiment includes one or more antibodies specific for these proteins and means for detecting the binding of the antibodies to these proteins by, e.g., a standard sandwich immunoassay as described herein. Where the presence of a protein within a cell is to be detected, e.g., as from a tissue sample, the kit also may comprise means for disrupting the cell structure so as to expose intracellular proteins.

2.B. Nucleic Acid-based Assays

The presence of a prostate cancer in an individual also may be determined by detecting, in a tissue or body fluid sample, a nucleic acid molecule encoding a prostate cancer-associated protein. Using methods well known to those of ordinary skill in the art, the prostate cancer-associated proteins of the invention may be sequenced, and then, based on the determined sequence, oligonucleotide probes designed for screening a cDNA library (see, for example, Sambrook et al. (1989), supra). associated target nucleic acid molecule encoding a marker prostate cancer-associated protein may be detected using a labeled binding moiety capable of specifically binding the target nucleic acid. The binding moiety may comprise, for example, a protein, a nucleic acid or a peptide nucleic acid. Additionally, a target nucleic acid, such as an mRNA encoding a prostate cancer-associated protein, may be detected by conducting, for example, a Northern blot analysis using labeled oligonucleotides, e.g., nucleic acid fragments complementary to and capable of hybridizing specifically with at least a portion of a target nucleic acid.

More specifically, gene probes comprising complementary RNA or, preferably, DNA to the prostate cancer-associated nucleotide sequences or mRNA sequences encoding prostate cancer-associated proteins may be produced using established recombinant techniques or oligonucleotide synthesis. The probes hybridize with complementary nucleic acid sequences presented in the test specimen, and can provide exquisite specificity. A short, well-defined probe, coding for a single unique sequence is most precise and preferred. Larger probes are generally less specific. While an oligonucleotide of any length may hybridize to an mRNA transcript, oligonucleotides typically within the range of 8-100 nucleotides, preferably within the range of 15-50 nucleotides, are envisioned to be most useful in standard hybridization assays. Choices of probe length and sequence allow one to choose the degree of specificity desired. Hybridization is carried out at from 50° to 65° C. in a high salt buffer solution, formamide or other agents to set the degree of complementarity required. Furthermore, the state of the art is such that probes can be manufactured to recognize essentially any DNA or RNA sequence. For additional particulars, see, for example, Berger et al. (1987) Guide to Molecular Techniques (Methods of Enzymology, vol. 152).

A wide variety of different labels coupled to the probes or antibodies may be employed in the assays. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking are taught in the literature. Broadly, the labels may be divided into the following categories: chromogens; catalyzed reactions; chemiluminescence; radioactive labels; and colloidal-sized colored particles. The chromogens include compounds which absorb light in a distinctive range so that a color may be observed, or emit light when irradiated with light of a particular wavelength or wavelength range, e.g., fluorescers. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

A common method of in vitro labeling of nucleotide probes involves nick translation wherein the unlabeled DNA probe is nicked with an endonuclease to produce free 3′hydroxyl termini within either strand of the double-stranded fragment. Simultaneously, an exonuclease removes the nucleotide residue from the 5′ phosphoryl side of the nick. The sequence of replacement nucleotides is determined by the sequence of the opposite strand of the duplex. Thus, if labeled nucleotides are supplied, DNA polymerase will fill in the nick with the labeled nucleotides. Using this well-known technique, up to 50% of the molecule can be labeled. For smaller probes, known methods involving 3′ end labeling may be used. Furthermore, there are currently commercially available methods of labeling DNA with fluorescent molecules, catalysts, enzymes, or chemiluminescent materials. Biotin labeling kits are commercially available (Enzo Biochem Inc.) under the trademark Bio-Probe. This type of system permits the probe to be coupled to avidin which in turn is labeled with, for example, a fluorescent molecule, enzyme, antibody, etc. For further disclosure regarding probe construction and technology, see, for example, Sambrook et al. (1989) supra, or Wu et al. (1997) Methods In Gene Biotechnology, CRC Press, New York.

The oligonucleotide selected for hybridizing to the target nucleic acid, whether synthesized chemically or by recombinant DNA methodologies, is isolated and purified using standard techniques and then preferably labeled (e.g., with ³⁵S or ³²P) using standard labeling protocols. A sample containing the target nucleic acid then is run on an electrophoresis gel, the dispersed nucleic acids transferred to a nitrocellulose filter and the labeled oligonucleotide exposed to the filter under stringent hybridizing conditions, e.g., 50% formamide, 5×SSPE, 2×Denhardt's solution, 0.1% SDS at 42° C., as described in Sambrook et al. (1989) supra. The filter may then be washed using 2×SSPE, 0. 1% SDS at 68° C., and more preferably using 0.1×SSPE, 0.1% SDS at 68° C. Other useful procedures known in the art include solution hybridization, and dot and slot RNA hybridization. Optionally, the amount of the target nucleic acid present in a sample is then quantitated by measuring the radioactivity of hybridized fragments, using standard procedures known in the art.

In addition, oligonucleotides also may be used to identify other sequences encoding members of the target protein families. The methodology also may be used to identify genetic sequences associated with the nucleic acid sequences encoding the proteins described herein, e.g., to identify non-coding sequences lying upstream or downstream of the protein coding sequence, and which may play a functional role in expression of these genes. Additionally, binding assays may be conducted to identify and detect proteins capable of a specific binding interaction with a nucleic acid encoding a prostate cancer-associated protein, which may be involved, e.g., in gene regulation or gene expression of the protein. In a further embodiment, the assays described herein may be used to identify and detect nucleic acid molecules comprising a sequence capable of recognizing and being specifically bound by a prostate cancer-associated protein.

In addition, it is anticipated that using a combination of appropriate oligonucleotide primers, i.e., more than one primer, the skilled artisan may determine the level of expression of a target gene in vivo by standard polymerase chain reaction (PCR) procedures, for example, by quantitative PCR. Conventional PCR based assays are discussed, for example, in Innes et al (1990) PCR Protocols: a Guide to Methods and Applications, Academic Press and Innes et al. (1995) PCR Strategies, Academic Press, San Diego, Calif.

Identification of Proteins which Interact in Vivo with Prostate Cancer-associated Proteins

In addition, it is contemplated that the skilled artisan, using procedures like those described hereinbelow, may identify other molecules which interact in vivo with the prostate cancer-associated proteins described herein. Such molecules also may provide possible targets for chemotherapy.

By way of example, cDNA encoding proteins or peptides capable of interacting with prostate cancer-associated proteins can be determined using a two-hybrid assay, as reported in Durfee et al. (1993) Genes & Develop. 7: 555-559. The principle of the two hybrid system is that noncovalent interaction of two proteins triggers a process (transcription) in which these proteins normally play no direct role, because of their covalent linkage to domains that function in this process. For example, in the two-hybrid assay, detectable expression of a reporter gene occurs when two fusion proteins, one comprising a DNA-binding domain and one comprising a transcription initiation domain, interact.

The skilled artisan can use a host cell that contains one or more reporter genes, such as yeast strain Y153, reported in Durfee et al. (1993) supra. This strain carries two chromosomally located reporter genes whose expression is regulated by Gal4. A first reporter gene, is the E. coli lacZ gene under the control of the Gal4 promoter. A second reporter gene is the selectable HIS3 gene. Other useful reporter genes may include, for example, the luciferase gene, the LEU2 gene, and the GFP (Green Fluorescent Protein) gene.

Two sets of plasmids are used in the two hybrid system. One set of plasmids contains DNA encoding a Gal4 DNA-binding domain fused in frame to DNA encoding a prostate cancer-associated protein. The other set of plasmids contain DNA encoding a Gal4 activation domain fused to portions of a human cDNA library constructed from human lymphocytes. Expression from the first set of plasmids results in a fusion protein comprising a Gal4 DNA-binding domain and a prostate cancer-associated protein. Expression from the second set of plasmids produces a transcription activation protein fused to an expression product from the lymphocyte cDNA library. When the two plasmids are transformed into a Gal4-deficient host cell, such as the yeast Y153 cells described above, interaction of the Gal4 DNA binding domain and transcription activation domain occurs only if the prostate cancer-associated protein fused to the DNA binding domain binds to a protein expressed from the lymphocyte cDNA library fused to the transcription activating domain. As a result of the protein-protein interaction between the prostate cancer-associated protein and its in vivo binding partner detectable levels of reporter gene expression occur.

In addition to identifying molecules which interact in vivo with the prostate cancer-associated proteins, the skilled artisan may also screen for molecules, for example, small molecules which alter or inhibit specific interaction between a prostate cancer-associated protein and its in vivo binding partner.

For example, a host cell can be transfected with DNA encoding a suitable DNA binding domain/prostate cancer-associated protein hybrid and a translation activation domain/putative prostate cancer-associated protein binding partner, as disclosed above. The host cell also contains a suitable reporter gene in operative association with a cis-acting transcription activation element that is recognized by the transcription factor DNA binding domain. The level of reporter gene expressed in the system is assayed. Then, the host cell is exposed to a candidate molecule and the level of reporter gene expression is detected. A reduction in reporter gene expression is indicative of the candidate's ability to interfere with complex formation or stability with respect to the prostate cancer-associated protein and its in vivo binding partner. As a control, the candidate molecule's ability to interfere with other, unrelated protein-protein complexes is also tested. Molecules capable of specifically interfering with a prostate cancer-associated protein/binding partner interaction, but not other protein-protein interactions, are identified as candidates for production and further analysis. Once a potential candidate has been identified, its efficacy in modulating cell cycling and cell replication can be assayed in a standard cell cycle model system.

Candidate molecules can be produced as described hereinbelow. For example, DNA encoding the candidate molecules can be inserted, using conventional techniques well described in the art (see, for example, Sambrook (1989) supra) into any of a variety of expression vectors and transfected into an appropriate host cell to produce recombinant proteins, including both full length and truncated forms. Useful host cells include E. coli, Saccharomyces cerevisiae, Pichia pastoris, the insect/baculovirus cell system, myeloma cells, and various other mammalian cells. The full length forms of such proteins are preferably expressed in mammalian cells, as disclosed herein. The nucleotide sequences also preferably include a sequence for targeting the translated sequence to the nucleus, using, for example, a sequence encoding the eight amino acid nucleus targeting sequence of the large T antigen, which is well characterized in the art. The vector can additionally include various sequences to promote correct expression of the recombinant protein, including transcription promoter and termination sequences, enhancer sequences, preferred ribosome binding site sequences, preferred mRNA leader sequences, preferred protein processing sequences, preferred signal sequences for protein secretion, and the like. The DNA sequence encoding the gene of interest can also be manipulated to remove potentially inhibiting sequences or to minimize unwanted secondary structure formation. As will be appreciated by the practitioner in the art, the recombinant protein can also be expressed as a fusion protein.

After translation, the protein can be purified from the cells themselves or recovered from the culture medium. The DNA can also include sequences which aid in expression and/or purification of the recombinant protein. The DNA can be expressed directly or can be expressed as part of a fusion protein having a readily cleavable fusion junction.

The DNA may also be expressed in a suitable mammalian host. Useful hosts include fibroblast 3T3 cells, (e.g., NIH 3T3, from CRL 1658) COS (simian kidney ATCC, CRL-1650) or CHO (Chinese hamster ovary) cells (e.g., CHO-DXB11, from Chasin (1980) Proc. Nat'l. Acad. Sci. USA 77 :4216-4222), mink-lung epithelial cells (MV1Lu), human foreskin fibroblast cells, human glioblastoma cells, and teratocarcinoma cells. Other useful eukaryotic cell systems include yeast cells, the insect/baculovirus system or myeloma cells-see, for example, Wu et al. (1997) supra.

In order to express a candidate molecule, the DNA is subcloned into an insertion site of a suitable, commercially available vector along with suitable promoter/enhancer sequences and 3′ termination sequences. Useful promoter/enhancer sequence combinations include the CMV promoter (human cytomegalovirus (MIE) promoter) present, for example, on pCDM8, as well as the mammary tumor virus promoter (MMTV) boosted by the Rous sarcoma virus LTR enhancer sequence (e.g., from Clontech, Inc., Palo Alto). A useful inducable promoter includes, for example, a Zn²⁺-inducible promoter, such as the Zn²⁺ metallothionein promoter (Wrana et al. (1992) Cell 71: 1003-1014). Other inducible promoters are well known in the art and can be used with similar success. Expression also can be further enhanced using trans-activating enhancer sequences. The plasmid also preferably contains an amplifiable marker, such as DHFR under suitable promoter control, e.g., SV40 early promoter (ATCC #37148). Transfection, cell culturing, gene amplification and protein expression conditions are standard conditions, well known in the art, such as are described, for example in Ausubel et al., eds., (1989) Current Protocols in Molecular Biology, John Wiley & Sons, NY. Briefly, transfected cells are cultured in medium containing 5-10% dialyzed fetal calf serum (dFCS), and stably transfected high expression cell lines obtained by amplification and subcloning and evaluated by standard Western and Northern blot analysis. Southern blots also can be used to assess the state of integrated sequences and the extent of their copy number amplification.

The expressed candidate protein is then purified using standard procedures. A currently preferred methodology uses an affinity column, such as a ligand affinity column or an antibody affinity column. The column then is washed, and the candidate molecules selectively eluted in a gradient of increasing ionic strength, changes in pH, or addition of mild detergent. It is appreciated that in addition to the candidate molecules which bind to the prostate cancer-associated proteins, the prostate cancer associated proteins themselves may likewise be produced using such recombinant DNA technologies.

Prostate Cancer Therapy and Methods for Monitoring Therapy

The skilled artisan, after identification of prostate cancer-associated proteins and proteins which interact with the prostate cancer-associated proteins, can develop a variety of therapies for treating prostate cancer. Because the marker proteins described herein are present at detectably higher levels in prostate cancer cells relative to normal prostate cells, the skilled artisan may employ, for example, the marker proteins and/or nucleic acids encoding the marker proteins as target molecules for a cancer chemotherapy.

4.A. Anti-sense-based Therapeutics

A particularly useful cancer therapeutic envisioned is an oligonucleotide or peptide nucleic acid sequence complementary and capable of hybridizing under physiological conditions to part, or all, of the gene encoding the marker protein or to part, or all, of the transcript encoding the marker protein thereby to reduce or inhibit transcription and/or translation of the marker protein gene. Alternatively, the same technologies may be applied to reduce or inhibit transcription and/or translation of the proteins that interact with the prostate cancer-associated proteins.

Anti-sense oligonucleotides have been used extensively to inhibit gene expression in normal and abnormal cells. For reviews of anti-sense theory and established protocols, see, for example, Phillips, ed. (1999) Antisense Technology (Methods in Enzymology, vols. 313-314), Academic Press; Hartmann, ed. (1999) Manual of Antisense Methodology, Kluwer Law International; Stein, ed. (1998) Applied Antisense Oligonucleotide Technology, Wiley-Liss; and Stein et al. (1988) Cancer Res. 48: 2659-2668. In addition, the synthesis and use of peptide nucleic acids as anti-sense-based therapeutics are described in PCT publications PCT/EP92/01219 published Nov. 26, 1992, PCT/US92/10921 published Jun. 24, 1993, and PCT/US94/013523 published Jun. 1, 1995. Accordingly, the anti-sense-based therapeutics may be used as part of chemotherapy, either alone or in combination with other therapies.

Anti-sense oligonucleotide and peptide nucleic acid sequences are capable of hybridizing to a gene and/or mRNA transcript and, therefore, may be used to inhibit transcription and/or translation of the protein described herein. It is appreciated, however, that oligoribonucleotide sequences generally are more susceptible to enzymatic attack by ribonucleases than are deoxyribonucleotide sequences. Hence, oligodeoxyribonucleotides are preferred over oligoribonucleotides for in vivo therapeutic use. It is appreciated that the peptide nucleic acid sequences, unlike regular nucleic acid sequences, are not susceptible to nuclease degradation and, therefore, are likely to have greater longevity in vivo. Furthermore, it is appreciated that peptide nucleic acid sequences bind complementary single stranded DNA and RNA strands more strongly than corresponding DNA sequences (see, for example, PCT/EP92/20702 published Nov. 26, 1992). Accordingly, peptide nucleic acid sequences are preferred for in vivo therapeutic use.

Therapeutically useful anti-sense oligonucleotides or peptide nucleic acid sequences may be synthesized by any of the known chemical oligonucleotide and peptide nucleic acid synthesis methodologies well known and thoroughly described in the art. Alternatively, a sequence complementary to part or all of the natural mRNA sequence may be generated using standard recombinant DNA technologies.

Because the complete nucleotide sequence encoding the entire marker protein as well as additional 5′ and 3′ untranslated sequences are known for each of the marker proteins and/or can be determined readily using techniques well known in the art, anti-sense oligonucleotides or peptide nucleic acids which hybridize with any portion of the mRNA transcript or non-coding sequences may be prepared using conventional oligonucleotide and peptide nucleic acid synthesis methodologies.

Oligonucleotides complementary to, and hybridizable with, any portion of the mRNA transcripts encoding the marker proteins are, in principle, effective for inhibiting translation of the target proteins as described herein. For example, as described in U.S. Pat. No. 5,098,890, oligonucleotides complementary to mRNA at or near the translation initiation codon site may be used to inhibit translation. Moreover, it has been suggested that sequences that are too distant in the 3′ direction from the translation initiation site may be less effective in hybridizing the mRNA transcripts because of potential ribosomal “read-through”, a phenomenon whereby the ribosome is postulated to unravel the anti-sense/sense duplex to permit translation of the message.

A variety of sequence lengths of oligonucleotide or peptide nucleic acid may be used to hybridize to mRNA transcripts. However, very short sequences (e.g., sequences containing less than 8-15 nucleobases) may bind with less specificity. Moreover, for in vivo use, short oligonucleotide sequences may be particularly susceptible to enzymatic degradation. Peptide nucleic acids, as mentioned above, likely are resistant to nuclease degradation. Where oligonucleotide and peptide nucleic acid sequences are to be provided directly to the cells, very long sequences may be less effective at inhibition because of decreased uptake by the target cell. Accordingly, where the oligonucleotide or peptide nucleic acid is to be provided directly to target cells, oligonucleotide and/or peptide nucleic acid sequences containing about 8-50 nucleobases, and more preferably 15-30 nucleobases, are envisioned to be most advantageous.

An alternative means for providing anti-sense oligonucleotide sequences to a target cell is gene therapy where, for example, a DNA sequence, preferably as part of a vector and associated with a promoter, is expressed constitutively inside the target cell. Oeller et al. (Oeller et al. (1992) Science 254: 437-539) describe the in vivo inhibition of the ACC synthase enzyme using a constitutively expressible DNA sequence encoding an anti-sense sequence to the full length ACC synthase transcript. Accordingly, where the anti-sense oligonucleotide sequences are provided to a target cell indirectly, for example, as part of an expressible gene sequence to be expressed within the cell, longer oligonucleotide sequences, including sequences complementary to substantially all the protein coding sequence, may be used to advantage.

Finally, therapeutically useful oligonucleotide sequences envisioned also include not only native oligomers composed of naturally occurring nucleotides, but also those comprising modified nucleotides, for example, to improve stability and lipid solubility and thereby enhance cellular uptake. For example, it is known that enhanced lipid solubility and/or resistance to nuclease digestion results by substituting a methyl group or sulfur atom for a phosphate oxygen in the internucleotide phosphodiester linkage. Phosphorothioates (“S-oligonucleotides” wherein a phosphate oxygen is replaced by a sulfur atom), in particular, are stable to nuclease cleavage, are soluble in lipids, and are preferred, particularly for direct oligonucleotide administration. S-oligonucleotides may be synthesized chemically using conventional synthesis methodologies well known and thoroughly described in the art.

Preferred synthetic internucleoside linkages include phosphorothioates, alkylphosphonates, phosphorodithioates, phosphate esters, alkylphosphonothioates, phosphoramidates, carbamates, carbonates, phosphate triesters, acetamidate, and carboxymethyl esters. Furthermore, one or more of the 5′-3′ phosphate group may be covalently joined to a low molecular weight (e.g., 15-500 Da) organic group, including, for example, lower alkyl chains or aliphatic groups (e.g., methyl, ethyl, propyl, butyl), substituted alkyl and aliphatic groups (e.g., aminoethyl, aminopropyl, aminohydroxyethyl, aminohydroxypropyl), small saccharides or glycosyl groups. Other low molecular weight organic modifications include additions to the internucleoside phosphate linkages such as cholesteryl or diamine compounds with varying numbers of carbon residues between the amino groups and terminal ribose. Oligonucleotides with these linkages or with other modifications can be prepared using methods well known in the art (see, for example, U.S. Pat. No. 5,149,798).

Suitable oligonucleotide and/or peptide nucleic acid sequences which inhibit transcription and/or translation of the marker proteins can be identified using standard in vivo assays well characterized in the art. Preferably, a range of doses is used to determine effective concentrations for inhibition as well as specificity of hybridization. For example, in the cases of an oligonucleotide, a dose range of 0-100 μg oligonucleotide/ml may be assayed. Further, the oligonucleotides may be provided to the cells in a single transfection, or as part of a series of transfections. Anti-sense efficacy may be determined by assaying a change in cell proliferation over time following transfection, using standard cell counting methodology and/or by assaying for reduced expression of marker protein, e.g., by immunofluorescence. Alternatively, the ability of cells to take up and use thymidine is another standard means of assaying for cell division and may be used here, e.g., using ³H-thymidine. Effective anti-sense inhibition should inhibit cell division sufficiently to reduce thymidine uptake, inhibit cell proliferation, and/or reduce detectable levels of marker proteins.

It is anticipated that therapeutically effective oligonucleotide or peptide nucleic acid concentrations may vary according to the nature and extent of the neoplasm, the particular nucleobase sequence used, the relative sensitivity of the neoplasm to the oligonucleotide or peptide nucleic acid sequence, and other factors. Useful ranges for a given cell type and oligonucleotide and/or peptide nucleic acid may be determined by performing standard dose range experiments. Dose range experiments also may be performed to assess toxicity levels for normal and malignant cells. It is contemplated that useful concentrations may range from about 1 to 100 μg/ml per 10⁵ cells.

For in vivo use, the anti-sense oligonucleotide or peptide nucleic acid sequences may be combined with a pharmaceutically acceptable carrier, such as a suitable liquid vehicle or excipient, and optionally an auxiliary additive or additives. Liquid vehicles and excipients are conventional and are available commercially. Illustrative thereof are distilled water, physiological saline, aqueous solutions of dextrose, and the like. For in vivo cancer therapies, the anti-sense sequences preferably can be provided directly to malignant cells, for example, by injection directly into the tumor. Alternatively, the oligonucleotide or peptide nucleic acid may be administered systemically, provided that the anti-sense sequence is associated with means for directing the sequences to the target malignant cells.

In addition to administration with conventional carriers, the anti-sense oligonucleotide or peptide nucleic acid sequences may be administered by a variety of specialized oligonucleotide delivery techniques. For example, oligonucleotides may be encapsulated in liposomes, as described in Mannino et al. (1988) BioTechnology 6: 682, and Felgner et al. (1989) Bethesda Res. Lab. Focus 11:21. Lipids useful in producing liposomal formulations include, without limitation, monoglycerides, diglycerides, sulfatides, lysolecithin, phospholipids, saponin, bile acids, and the like. Preparation of such liposomal formulations is within the level of skill in the art (see, for example, in U.S. Pat. Nos. 4,235,871; 4,501,728; 4,837,028; and 4,737,323). The pharmaceutical composition of the invention may further include compounds such as cyclodextrins and the like which enhance delivery of oligonucleotides into cells. When the composition is not administered systemically but, rather, is injected at the site of the target cells, cationic detergents (e.g. Lipofectin) may be added to enhance uptake. In addition, reconstituted virus envelopes have been successfully used to deliver RNA and DNA to cells (see, for example, Arad et al. (1986) Biochem. Biophy. Acta. 859: 88-94).

For therapeutic use in vivo, the anti-sense oligonucleotide and/or peptide nucleic acid sequences are administered to the individual in a therapeutically effective amount, for example, an amount sufficient to reduce or inhibit target protein expression in malignant cells. The actual dosage administered may take into account whether the nature of the treatment is prophylactic or therapeutic in nature, the age, weight, health of the patient, the route of administration, the size and nature of the malignancy, as well as other factors. The daily dosage may range from about 0.01 to 1,000 mg per day. Greater or lesser amounts of oligonucleotide or peptide nucleic acid sequences may be administered, as required. As will be appreciated by those skilled in the medical art, particularly the chemotherapeutic art, appropriate dose ranges for in vivo administration would be routine experimentation for a clinician. As a preliminary guideline, effective concentrations for in vitro inhibition of the target molecule may be determined first.

4.B. Binding Protein-based Therapeutics.

As mentioned above, a cancer marker protein or a protein that interacts with the cancer marker protein may be used as a target for chemotherapy. For example, a binding protein designed to bind the marker protein essentially irreversibly can be provided to the malignant cells, for example, by association with a ligand specific for the cell and known to be absorbed by the cell. Means for targeting molecules to particular cells and cell types are well described in the chemotherapeutic art.

Binding proteins may be obtained and tested using technologies well known in the art. For example, the binding portions of antibodies may be used to advantage. It is contemplated, however, that intact antibodies or BABS that have preferably been humanized may be used in the practice of the invention. As used herein, the term “humanized” is understood to mean a process whereby the framework region sequences of a non-human immunoglobulin variable region are replaced by corresponding human framework sequences. Accordingly, it is contemplated that such humanized binding proteins will elicit a weaker immune response than their unhumanized counterparts. Particularly useful are binding proteins identified with high affinity for the target protein, e.g., greater than about 10⁹ M⁻¹ Alternatively, DNA encoding the binding protein may be provided to the target cell as part of an expressible gene to be expressed within the cell following the procedures used for gene therapy protocols well described in the art. See, for example, U.S. Pat. No. 4,497,796, and Baichwal, ed. (1986) Gene Transfer. It is anticipated that, once bound by binding protein, the target protein will be inactivated or its biological activity reduced thereby inhibiting or retarding cell division.

As described above, suitable binding proteins for in vivo use may be combined with a suitable pharmaceutically-acceptable carrier, such as physiological saline or other useful carriers well characterized in the medical art. The pharmaceutical compositions may be provided directly to malignant cells, for example, by direct injection, or may be provided systemically, provided the binding protein is associated with means for targeting the protein to target cells. Finally, suitable dose ranges and cell toxicity levels may be assessed using standard dose range experiments. Therapeutically-effective concentrations may range from about 0.01 to about 1,000 mg per day. As described above, actual dosages administered may vary depending, for example, on the nature of the malignancy, the age, weight and health of the individual, as well as other factors.

4.C. Small Molecule-based Therapeutics.

After having isolated prostate cancer-associated proteins, the skilled artisan can, using methodologies well known in the art, screen small molecule libraries (either peptide or non-peptide based libraries) to identify candidate molecules that reduce or inhibit the biological function of the prostate cancer-associated proteins. The small molecules preferably accomplish this function by reducing the in vivo expression of the target molecule, or by interacting with the target molecule thereby to inhibit either the biological activity of the target molecule or an interaction between the target molecule and its in vivo binding partner.

It is contemplated that, once the candidate small molecules have been elucidated, the skilled artisan may enhance the efficacy of the small molecule using rational drug design methodologies well known in the art. Alternatively, the skilled artisan may use a variety of computer programs which assist the skilled artisan to develop quantitative structure activity relationships (QSAR) which further to assist the design of additional candidate molecules de novo. Once identified, the small molecules may be produced in commercial quantities and subjected to the appropriate safety and efficacy studies.

It is contemplated that the screening assays may be automated thereby facilitating the screening of a large number of small molecules at the same time. Such automation procedures are within the level of skill in the art of drug screening and, therefore, are not discussed herein. Candidate peptide-based small molecules may be produced by expression of an appropriate nucleic acid sequence in a host cell or using synthetic organic chemistries. Similarly, non-peptidyl-based small molecules may be produced using conventional synthetic organic chemistries well known in the art.

As described above, for in vivo use, the identified small molecules may be combined with a suitable pharmaceutically acceptable carrier, such as physiological saline or other useful carriers well characterized in the medical art. The pharmaceutical compositions may be provided directly to malignant cells, for example, by direct injection, or may be provided systemically, provided the binding protein is associated with means for targeting the protein to target cells. Finally, suitable dose ranges and cell toxicity levels may be assessed using standard dose range experiments. As described above, actual dosages administered may vary depending, for example, on the nature of the malignancy, the age, weight and health of the individual, as well as other factors.

4.D. Methods for Monitoring the Status of Prostate Cancer in an Individual

The progression of the prostate cancer or the therapeutic efficacy of chemotherapy may be measured using procedures well known in the art. For example, the efficacy of a particular chemotherapeutic agent can be determined by measuring the amount of a prostate cancer-associated protein released from prostate cancer cells undergoing cell death. As reported in U.S. Pat. Nos. 5,840,503 and 5,965,376, soluble nuclear matrix proteins and fragments thereof are released by cells upon cell death. Such soluble nuclear matrix proteins can be quantitated in a body fluid and used to monitor the degree or rate of cell death in a tissue. Similarly, the levels of one or more prostate cancer-associated proteins could be used as an indication of the status of prostate cancer in the individual.

For example, the concentration of a prostate cancer-associated protein or a fragment thereof released from cells is compared to standards from healthy, untreated tissue. Fluid samples are collected at discrete intervals during treatment and compared to the standard. It is contemplated that changes in the level of the prostate cancer-associated protein, for example, will be indicative of the efficacy of treatment (that is, the rate of cancer cell death). It is contemplated that the release of soluble, prostate cancer-associated proteins can be measured in blood, plasma, urine, sputum, semen, seminal fluid, seminal plasma, prostatic secretion and other body fluids.

Where the assay is used to monitor tissue viability or progression of prostate cancer, the step of detecting the presence and abundance of the marker protein or its transcript in samples of interest is repeated at intervals and these values then are compared, the changes in the detected concentrations reflecting changes in the status of the tissue. For example, an increase in the level of one or more prostate cancer-associated proteins may correlate with progression of the prostate cancer. Where the assay is used to evaluate the efficacy of a therapy, the monitoring steps occur following administration of the therapeutic agent or procedure (e.g., following administration of a chemotherapeutic agent or following radiation treatment). Similarly, a decrease in the level of prostate cancer-associated proteins may correlate with a regression of the prostate cancer.

Thus, prostate cancer may be identified by the presence of prostate cancer-associated proteins as taught herein. Once identified, the prostate cancer may be treated using compounds that reduce in vivo the expression and/or biological activity of the prostate cancer-associated proteins. Furthermore, the methods provided herein can be used to monitor the progression and/or treatment of the disease. The following non-limiting examples provide details of the isolation and characterization of prostate cancer-associated proteins and methods for their use in the detection of prostate cancer.

### EXAMPLE 1

Identification of Prostate Cancer Markers

To identify markers for prostate cancer, the sera of individuals with prostate cancer were compared to the sera of normal individuals by surface-enhanced laser desorption and ionization (SELDI) mass spectrometry. Briefly, 0.5 mL aliquots of sera harvested from the individuals were thawed. Then, 1 μL of a 1 mg/mL solution of soybean trypsin inhibitor (SBTI) and 1 μL of a 1 mg/mL solution of pepstatin were added to each aliquot. To remove lipids, 350 μl of 1,1,2-trifluorotrichloroethane was added to each sample. The samples then were vortexed for five minutes and centrifuged in a microcentrifuge for five minutes at 4° C. The resulting supernatants were applied to a 1 mL column of agarose coupled to protein G (Hitrap Protein G column, Pharmacia and Upjohn, Peapack, N.J.) to remove immunoglobulin proteins. The column then was rinsed with 3 mL of 50 mM sodium phosphate, pH 7.0, with SBTI and pepstatin (“binding buffer”), and the resulting flowthrough applied directly to a 5 mL column of 6% Sepharose coupled to Cibacron blue (Hitrap blue column, Pharmacia and Upjohn, Peapack, N.J.) to remove albumin proteins. The Hitrap blue column was rinsed with 10 mL of binding buffer. The resulting flow-through was concentrated using four centrifugation-based concentrators with a 10 kD cutoff (Centricon 10, Millipore Corporation, Bedford, Mass.) to a final volume of about 0.3 mL.

The resulting serum (substantially free of immunoglobulin and albumin) was subdivided into fourteen fractions containing approximately equal amounts of protein by ion exchange high pressure liquid chromatography (HPLC). Specifically, the serum was applied to a Protein Pak Q 8HR ion exchange column from Waters (catalog number WAT039575) in 50 mM sodium phosphate buffer, pH 7.0 and proteins were eluted from the column by increasing the concentration of sodium chloride in a stepwise manner. Thus, the serum was divided into fourteen fractions based on the concentration of sodium chloride used for elution. These fractions, accordingly, were designated: 0 mM, 25 mM, 50 mM, 75 mM, 100 mM, 125 mM, 150 mM, 175 mM, 200 mM, 225 mM, 250 mM, 300 mM, 400 mM, and 1M sodium chloride. After elution, each fraction was concentrated to approximately 100 μg/mL and buffer exchanged into binding buffer.

Ten μL from each of the fourteen fractions were applied and allowed to bind to each of four SELDI chip surfaces, each surface holding up to eight samples. The intended location of each sample on the chip was marked with a circle drawn using a hydrophobic marker like those used in Pap smears. The SELDI chips used herein were purchased from Ciphergen Biosystems, Inc., Palo Alto, Calif., and used as described below.

For nickel surfaces, a chip containing ethylenediaminetriacetic acid moieties (IMAC3, Ciphergen Biosystems, Inc., Palo Alto, Calif.), after use of the pen, was pretreated with two five-minute applications of 5 μL of a nickel salt solution, and washed with deionized water. After a five-minute treatment with 5 μL of binding buffer, 5 μL of sample were applied to the surface, which was left in a humidity chamber for one hour. The sample was removed by gently tapping the chip on a paper towel. Another 5 μL of sample were then applied, which was left in a humidity chamber for an additional sixty minutes. The chips then were washed twice: once with binding buffer to remove unbound proteins and once with deionized water to remove excess salts. 0.5 μL of sinapinic acid (12.5 mg/mL) was added twice and allowed to dry each time. The presence of sinapinic acid enhances the vaporization and ionization of the bound proteins in laser desorption/ionization mass spectrometry.

For hydrophobic chip surfaces (H4, Ciphergen Biosystems, Inc., Palo Alto, Calif., part number C5303-0028-04/Format A-H), after use of the pen, the surfaces was washed sequentially with 5 μL of 100% acetonitrile and with 5 μL of 50% acetonitrile. 5 μL of sample were added to the chip, which was left in a humidity chamber for one hour. The sample was removed by gently tapping the chip on a paper towel. An additional 5 μL of sample were added followed by another hour incubation in a humidity chamber. The chip was washed twice with 5 μL deionized water and treated with two applications of 0.5 μL sinapinic acid as above.

For chip surfaces containing carboxyl moieties (WCX-2, Ciphergen Biosystems, Inc., Palo Alto, Calif.), before use of the hydrophobic pen, the surface was washed with 10 mM HCl for thirty minutes and rinsed five times with deionized water. After use of the pen, the surface was washed five times with five μL of binding buffer and once with deionized water. Five μL of sample were added to the chip, which was left in a humidity chamber for one hour. The sample was removed by gently tapping the chip on a paper towel. An additional 5 μL of sample were added followed by another hour incubation in a humidity chamber. The surface was washed twice with 5 μL of binding buffer, and 0.5 μL of sinapinic acid were applied twice.

For chip surfaces containing quarternary ammonium moieties (SAX-2, Ciphergen Biosystems, Inc., Palo Alto, Calif.), after use of the pen, the surface was washed five times with 5 μL of binding buffer and once with deionized water. Application of sample, washing, and application of sinapinic acid were done as described above.

The chips then were subjected to mass spectrometry utilizing a Ciphergen SELDI PBS-1 (Ciphergen Biosystems, Inc., Palo Alto, Calif.) running the software program “SELDI v. 2.0b”. For all chips, “high mass” was set to 200,000 Daltons, “starting detector sensitivity” was set to 9 (from a range of 1-10, with 10 being the highest sensitivity), NDF (neutral density filter) was set to “OUT”, data acquisition method was set to “Seldi Quantitation”, SELDI acquisition parameters were set to 20 (except for H4, for which SELDI acquisition parameters were set to 21), with increments of 5, and warming with two shots at intensity 50 (out of 100) was included. For IMAC chips, mass was optimized from 3,000 Daltons to 50,000 Daltons, starting laser intensity was set to 80 (out of 100), and transients set to 5 (i.e., 5 laser shots per site). Peaks were identified automatically by the computer. For WCX-2 chips, mass was optimized from 3,000 Daltons to 50,000 Daltons, starting laser intensity was set to 80, and transients set to 8. Peaks were identified automatically by the computer. For SAX-2 chips, mass was optimized from 3,000 Daltons to 50,000 Daltons, starting laser intensity was set to 85, and transients set to 8. Peaks were identified automatically by the computer. For H4 chips, mass was optimized from 3,000 Daltons to 50,000 Daltons, starting laser intensity was set to 80, and transients were set to 5.

Thirty-six serum samples (sixteen from normal males and twenty from males with prostate cancer) were analyzed by mass spectrometry to identify the proteins present in the fifty-six fractions described above. The normal males were further classified as healthy based on clinical determination (PSA<2 ng/mL and unremarkable digital exam; N=10) or based on self-identification as healthy (N=6). The males with prostate cancer were further classified based on whether the cancer was confined to the prostate (N=2), not staged (N=5), or showed capsular penetration (N=13).

The resulting peaks in the mass spectrometry trace were compared to identify those peaks present in the serum samples from males with prostate cancer but not present in the normal samples. If peaks in different samples had a mass difference of no more than one percent, the peaks were presumed to be the same. Eight mass spectrometry peaks ranging in size from approximately 21,000 Da to approximately 60,000 Da were identified as present in at least eight samples from males with prostate cancer and in no more than two of the samples from normal males. The presence or absence of these peaks in samples from healthy females was also determined (N=4).

The results of the foregoing analyses are summarized in Table 1. The masses listed in the table are presumed accurate to within one percent.

TABLE 1

| | | | Number of | Number of | Number of | Number of | | |
|---|---|---|---|---|---|---|---|---|
| | | | positive | positive | positive | positive | Number of | |
| | | | samples | samples | samples from | samples | positive | Number of |
| | | | from males | from males | males with | from males | samples | positive |
| | Protein Pak | | with organ- | with | prostate cancer | clinically | from | samples |
| | Q 8 HR | SELDI | confined | prostate | showing | determined | males self- | from |
| | fraction (mM | chip | prostate | cancer of | capsular | to be | identified | healthy |
| | sodium | surface | cancer | unknown | penetration | healthy | as healthy | females |
| Mass (Da) | chloride) | used | (N = 2) | stage (N = 5) | (N = 13) | (N = 10) | (N = 6) | (N = 4) |
| 21310 | 125 mM | H4 | 0 | 5 | 12 | 2 | 0 | 3 |
| 22760 | 175 mM | Nickel | 0 | 2 | 11 | 0 | 0 | 0 |
| 25490 | 125 mM | WCX-2 | 1 | 4 | 8 | 0 | 0 | 0 |
| 25550 | 100 mM | H4 | 1 | 5 | 9 | 1 | 1 | 3 |
| 50760 | 125 mM | WCX-2 | 2 | 3 | 13 | 1 | 1 | 3 |
| 50710 | 125 mM | Nickel | 1 | 5 | 8 | 0 | 0 | 0 |
| 59660 | 0 mM | Nickel | 2 | 5 | 10 | 1 | 1 | 1 |
| | (flow-through) | | | | | | | |
| | and | | | | | | | |
| | 25 mM | | | | | | | |
| 126830 | 125 mM | Nickel | 0 | 2 | 6 | 0 | 0 | 0 |

### EXAMPLE 2

Analysis of 50.76 kD Protein as Prostate Cancer Marker

The effectiveness of the 50.76 kD protein identified as a prostate cancer marker in Example 1 was verified using a larger number of samples. Briefly, serum from additional patients known to have prostate cancer, or known to be healthy based on a negative digital rectal exam and prostate specific antigen levels below 2 ng/mL, was treated and fractionated as described above, and the 125 mM sodium chloride fractions were analyzed by SELDI using a WCX-2 chip as described above. The results from this expanded data set, which includes the data shown in Example 1, are depicted in Table 2.

TABLE 2

| Patient status | Incidence of 50.76 kD protein |
|---|---|
| Healthy | 2/50 (4%) |
| Prostate cancer (organ-confined) | 9/9 (100%) |
| Prostate cancer (capsular penetration) | 41/43 (95%) |
| Prostate cancer (all) | 50/52 (96%) |

Prostate specific antigen (PSA) levels in the serum were also analyzed to determine if the 50.76 kD protein can be used as a marker for cancers that would not be detected by a PSA assay. The results are shown in Table 3. From the data presented in Table 3, the 50.76 kD can be used to identify prostate cancer in samples which have been considered to be negative based on the PSA assay (shown in bold and italics). More specifically, the presence of the 50.76 kD protein can be used to identify cancer in individuals having a PSA concentration of less than 4 mg/L.

TABLE 3

| Cancer Sera Samples | | | | Healthy Sera Samples | | |
|---|---|---|---|---|---|---|
| | | | Presence | | | Presence |
| | | | of 50.8 | | | of 50.8 |
| | PSA | Cancer | kDa Peak | | PSA | kDa Peak |
| Number | (mg/L) | Stage | (+ or −) | Number | (mg/L) | (+ or −) |
| 1. | 0.9 | Cap. Pen. | + | 1. | 0.2 | − |
| 2. | 1.5 | Cap. Pen. | + | 2. | 0.2 | − |
| 3. | 1.5 | Org. Con. | + | 3. | 0.2 | − |
| 4. | 1.6 | Cap. Pen. | + | 4. | 0.3 | − |
| 5. | 1.7 | Cap. Pen. | − | 5. | 0.3 | − |
| 6. | 1.9 | Cap. Pen. | + | 6. | 0.4 | − |
| 7. | 2.2 | Cap. Pen. | + | 7. | 0.4 | − |
| 8. | 2.3 | Cap. Pen. | + | 8. | 0.4 | − |
| 9. | 3.7 | Org. Con. | + | 9. | 0.5 | − |
| 10. | 3.8 | Cap. Pen. | + | 10. | 0.5 | − |
| 11. | 3.9 | Cap. Pen. | + | 11. | 0.5 | − |
| 12. | 4.1 | Cap. Pen. | + | 12. | 0.5 | − |
| 13. | 4.1 | Org. Con. | + | 13. | 0.6 | − |
| 14. | 4.1 | Cap. Pen. | + | 14. | 0.6 | − |
| 15. | 4.3 | Cap. Pen. | + | 15. | 0.6 | − |
| 16. | 4.3 | Cap. Pen. | + | 16. | 0.6 | − |
| 17. | 4.4 | Cap. Pen. | + | 17. | 0.7 | − |
| 18. | 4.5 | Cap. Pen. | + | 18. | 0.7 | − |
| 19. | 4.8 | Cap. Pen. | + | 19. | 0.7 | − |
| 20. | 5.1 | Cap. Pen. | + | 20. | 0.7 | − |
| 21. | 5.1 | Org. Con. | + | 21. | 0.7 | − |
| 22. | 5.3 | Cap. Pen. | + | 22. | 0.7 | − |
| 23. | 5.5 | Org. Con. | + | 23. | 0.7 | − |
| 24. | 5.5 | Cap. Pen. | − | 24. | 0.7 | + |
| 25. | 5.7 | Cap. Pen. | + | 25. | 0.8 | − |
| 26. | 5.8 | Cap. Pen. | + | 26. | 0.8 | − |
| 27. | 5.9 | Cap. Pen. | + | 27. | 0.8 | − |
| 28. | 6.7 | Cap. Pen. | + | 28. | 0.8 | − |
| 29. | 7.0 | Org. Con. | + | 29. | 0.9 | − |
| 30. | 7.1 | Cap. Pen. | + | 30. | 0.9 | − |
| 31. | 7.2 | Cap. Pen. | + | 31. | 0.9 | − |
| 32. | 8.9 | Cap. Pen. | + | 32. | 1.0 | − |
| 33. | 9.1 | Cap. Pen. | + | 33. | 1.1 | − |
| 34. | 9.2 | Org. Con. | + | 34. | 1.1 | − |
| 35. | 9.7 | Org. Con. | + | 35. | 1.1 | − |
| 36. | 9.7 | Org. Con. | + | 36. | 1.1 | − |
| 37. | 9.8 | Cap. Pen. | + | 37. | 1.2 | − |
| 38. | 10.3 | Cap. Pen. | + | 38. | 1.2 | − |
| 39. | 10.5 | Cap. Pen. | + | 39. | 1.3 | − |
| 40. | 11.9 | Cap. Pen. | + | 40. | 1.4 | − |
| 41. | 12.3 | Cap. Pen. | + | 41. | 1.4 | − |
| 42. | 13.6 | Cap. Pen. | + | 42. | 1.4 | − |
| 43. | 16.5 | Cap. Pen. | + | 43. | 1.5 | − |
| 44. | 19.1 | Cap. Pen. | + | 44. | 1.6 | − |
| 45. | 22.0 | Cap. Pen. | + | 45. | 1.6 | − |
| 46. | 23.4 | Cap. Pen. | + | 46. | 1.7 | − |
| 47. | 26.6 | Cap. Pen. | + | 47. | 1.8 | − |
| 48. | — | Cap. Pen. | + | 48. | 1.9 | − |
| 49. | — | Cap. Pen. | + | 49. | 1.9 | + |
| 50. | 1.3 | Cap. Pen. | + | 50. | 1.9 | − |
| 51. | 7.1 | Cap. Pen. | + | | | |
| 52. | 12.3 | Cap. Pen. | + | | | |

Serum samples from twenty individuals with benign prostatic hyperplasia (BPH) were analyzed. The data are summarized in Table 4. The results indicate the presence of the 50.76 kD protein in 5 out of 20 individuals with BPH.

TABLE 4

| | | Presence of |
|---|---|---|
| | PSA | 50.8 kDa |
| Number | (mg/L) | Peak (+ or −) |
| 1. | 1.1 | − |
| 2. | 2.0 | − |
| 3. | 2.8 | + |
| 4. | 2.8 | − |
| 5. | 3.0 | + |
| 6. | 3.2 | − |
| 7. | 5.4 | − |
| 8. | 5.4 | − |
| 9. | 6.0 | − |
| 10. | 6.2 | − |
| 11. | 6.2 | − |
| 12. | 6.6 | + |
| 13. | 6.8 | + |
| 14. | 7.0 | − |
| 15. | 7.5 | − |
| 16. | 15.8 | − |
| 17. | — | − |
| 18. | — | + |
| 19. | — | − |
| 20. | — | − |

Similarly, serum samples from ten individuals from whom a prostate cancer had been removed were analyzed. The 50.76 kD protein was detected in the appropriate fraction in only two of the ten individuals, suggesting that production of the marker may cease or be reduced once the cancer is removed (Table 5). It is unknown whether metastasis occurred in any of these patients. If the cancer metastasized prior to surgery, the surgery would unlikely remove all of the cancer cells, which may explain the twenty percent incidence of the marker post-surgery.

TABLE 5

| | | Presence of |
|---|---|---|
| | PSA | 50.8 kDa |
| Number | (mg/L) | Peak (+ or −) |
| 1. | 0.5 | + |
| 2. | 0.3 | − |
| 3. | 0.0 | − |
| 4. | 0.0 | − |
| 5. | 0.0 | − |
| 6. | 0.1 | + |
| 7. | 0.02 | − |
| 8. | 0.02 | − |
| 9. | 0.2 | − |
| 10. | 0.1 | − |

### EXAMPLE 3

Purification and Characterization of 50.76 kD Prostate Cancer Protein

The 50.76 kD prostate cancer protein identified in Example 1 was isolated and further characterized as follows.

Approximately 26 mL of serum (combined from multiple prostate cancer patients) was depleted of immunoglobulin G and serum albumin using Protein G chromatography (30 mL column) and Affi-Blue agarose chromatography (250 mL column), respectively, using standard methodologies such as those described in Example 1. The albumin and immunoglobulin depleted serum then was fractionated by Mono Q ion-exchange affinity chromatography. Briefly, the serum proteins were applied to an HPLC system with a Protein Pak Q 8HR ion exchange column from Waters (catalog number WAT035980) in 50 mM sodium phosphate buffer, pH 7.0. The proteins were eluted from the column by increasing the concentration of sodium chloride in a stepwise manner. The fractions included flow through, and elution buffers of 50 mM sodium phosphate buffer, pH 7.0 containing 70 mM, 100 mM, 130 mM, 180 mM, and 1 M sodium chloride.

The eluted fractions then were concentrated by means of a Minicon 10 (Millipore) and exchanged into the binding buffer. The desalted 130 mM NaCl fraction from the HPLC step discussed above was subjected to sodium dodecyl sulfate-polyacrylamide gel electrophoresis (SDS-PAGE) using a 10% Tris-glycine gel. After electrophoresis, the resulting gel then was stained with Coomassie Brilliant Blue dye and destained to reveal the presence of proteins. The 50.8 kD band was excised from the acrylamide and treated with trypsin; the resulting peptides were eluted from the acrylamide slice and analyzed by liquid chromatography-mass spectrometry (LC-MS).

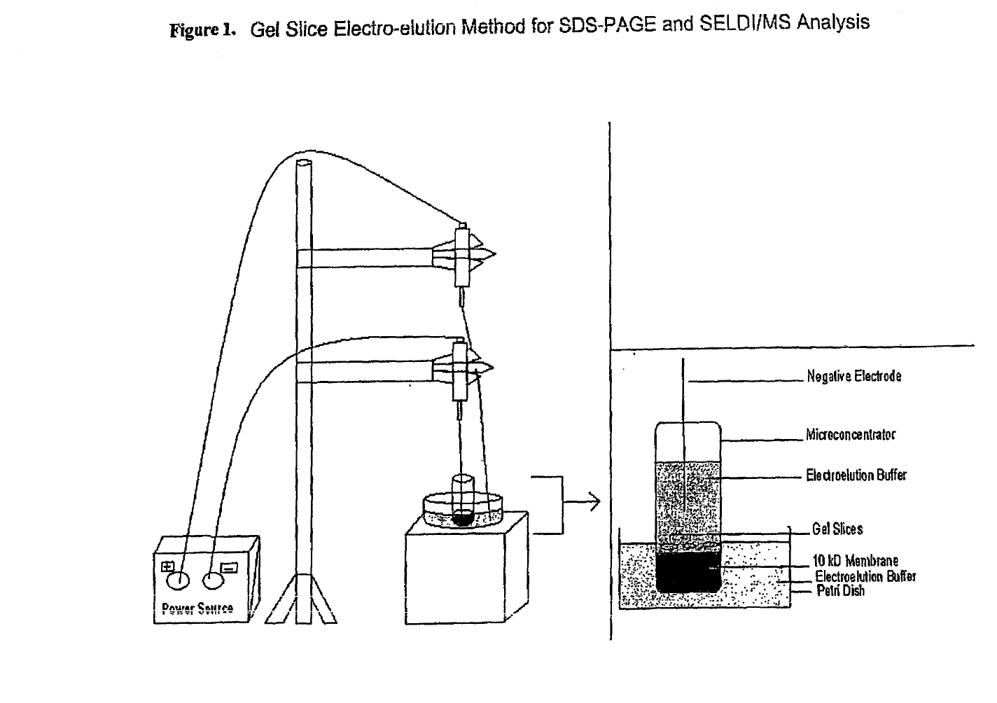

To verify that the 50.8 kD band did indeed include the 50.76 kD marker, a gel slice that was not treated with trypsin was subjected to electroelution to elute the intact protein from the gel. The gel slice was incubated in 1 M Tris-HCl, pH 8.0 for ten minutes with shaking at room temperature. The slice then was washed three times in distilled, deionized water and incubated for forty minutes in a solution of 100 μM NaOH. The slice then was cut into small pieces and incubated in an electroelution buffer (50 mM Tris, 100 mM NaCl, 0.2 M glycine, pH 8.5 inside a microfilterfuge tube (0.45 μM Nylon-66 filter from Rainin, catalog number 7016-022) with a membrane for retaining proteins larger than 10 kD. As shown in FIG. 1, the microfilterfuge tube was placed with its “downstream” portion in a Petri dish containing electroelution buffer; a positive electrode was placed in contact with the electroelution buffer in the Petri dish, and a negative electrode was placed in contact with the electroelution buffer in the “upstream” portion of the microfilterfuge tube. After 10 minutes of pre-incubation, a 3 mA current was applied for 18 hours. The protein successfully eluted into the electroelution buffer in the microfilterfuge tube.

The protein then was incubated overnight at 4° C. in 15 mM ammonium bicarbonate and buffer exchanged five times into 50 mM sodium phosphate. The resulting protein was detected by electrophoresis of 10 μL/lane on a 10% Tris-Glycine SD S-PAGE gel at 30 mA for 1 hour followed by silver staining. Two aliquots of 5 μL were also spotted onto a WCX-2 chip and analyzed by SELDI. SELDI analysis confirmed that the purified protein to be analyzed by LC-MS was indeed the 50.76 kD protein.

The peptide masses detected by LC-MS were compared to predicted masses for peptides from known proteins. Twelve detected masses corresponded to predicted masses for fragments of Vitamin D binding protein (VDBP; also known as Gc protein); the sequences of the corresponding fragments are shown in Table 6.

TABLE 6

| SEQ ID NO: | SEQUENCE: |
|---|---|
| 1 | FPSGTFEQVSQLVK |
| 2 | KFPSGTFEQVSQLVK |
| 3 | TSALSAK |
| 4 | EGLER |
| 5 | HLSLLTTLSNR |
| 6 | LCDNLSTK |
| 7 | YTFELSR |
| 8 | VMDKYTFELSR |
| 9 | THLPEVFLSK |
| 10 | RTHLPEVFLSK |
| 11 | VLEPTLK |
| 12 | GPLLK |

More than 120 genetic variants of VDBP have been identified. The three most common alleles are GC1F, GC1S and GC2. The DNA sequence of an exemplary allele and the amino acid sequence it encodes are depicted in FIG. 2. Protein fragments corresponding to the detected fragments of the 50.76 kD protein are underlined. A discussion of VDBP alleles and biology can be found in Braun et al. (1992) Human Genetics 89:401-406 and references cited therein, each of which is expressly incorporated herein by reference.

### EXAMPLE 4

Verification of the Identity of the 50.76 kD Protein as a VDBP-related Protein

A series of experiments was conducted to verify that the 50.76 kD protein is related to VDBP. First, the affinity of the 50.76 kD protein for Vitamin D was tested. The 125 mM fraction from the serum of a patient with cancer was placed in a Vitamin D-coated well from Biomedica. The sample was loaded into a well and incubated from 10 minutes. The sample then was removed and placed in a second well and incubated for an additional 10 minutes. This process was repeated until the sample had passed through 8 wells. After the final well, the sample was removed and tested for the presence of the 50.76 kD protein by SELDI. The 50.76 kD protein was not detectable, suggesting that the protein was indeed depleted by the contact with the Vitamin D-coated wells. The wells were washed with 50 mM phosphate buffer, pH 7.0. SELDI analysis of the eluate revealed the presence of a small amount of the 50.76 kD protein, further suggesting an affinity of the protein for the Vitamin-D coated wells.

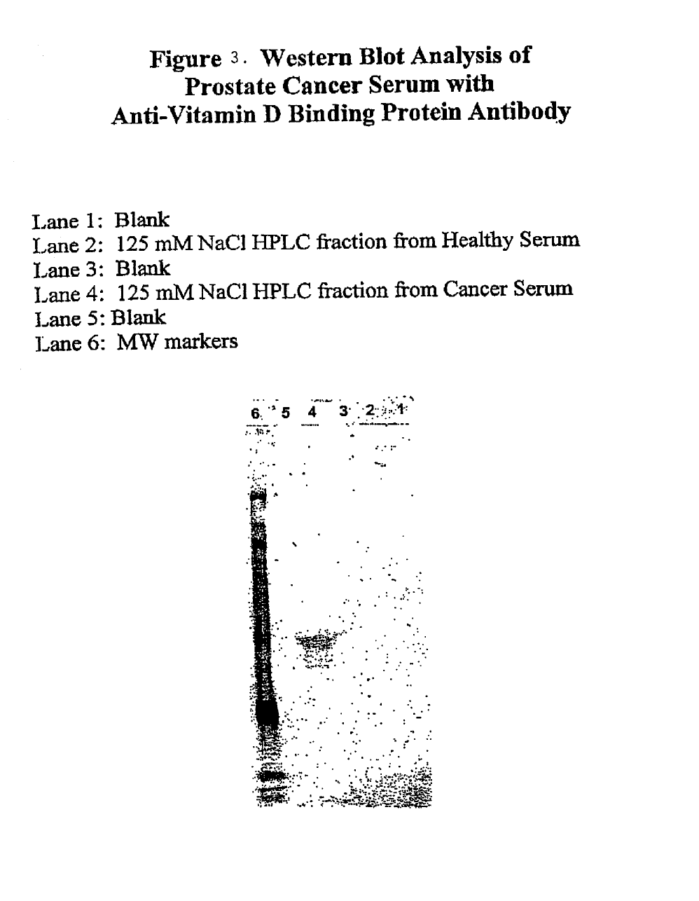

125 mM HPLC fractions (see Example 1) from males with and without prostate cancer were subjected to a Western blot and probed with a polyclonal antibody to VDBP (Strategic Biosolutions, Ramona, Calif.: Item Number S4103GND1-D0) or a monoclonal antibody to VDBP (Antibodyshop, Copenhagen, Denmark: Item Number HYB 249-01) using standard procedures. The results using the polyclonal anti-VDBP antibody are shown in FIG. 3. As shown, a protein is detected by the antibody in the fraction from a man with prostate cancer, but not in the fraction from a man without prostate cancer.

When the initial LC-MS experiments were done (see Example 3), three proteins were identified. The major protein, as discussed above, was VDBP. However, six peptides from anti-α-1-antitrypsin and two peptides from fetuin were also identified. While VDBP was the dominant protein in the 50.76 kD gel band (see Example 3), with the other proteins believed to be contaminants from other bands on the gel, it was necessary to verify that the 50.76 kD protein peak observed on the SELDI is indeed only due to a variant of VDBP. To do this, the following experiments were performed.

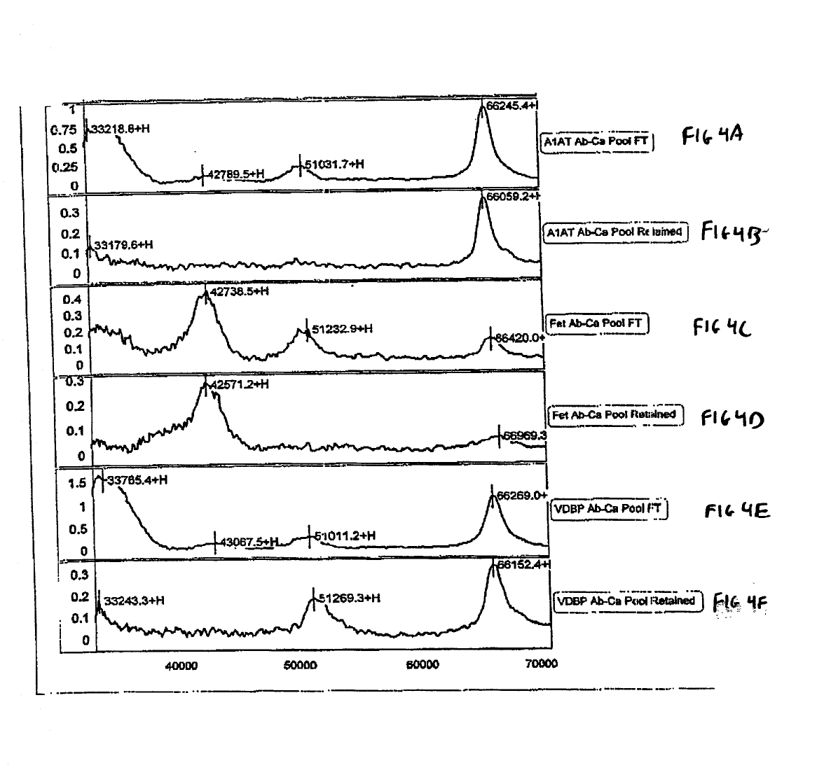

Wells containing Protein G were coated with the polyclonal anti-VDBP antibody, with an anti-fetuin antibody, or with an anti-α-1-antitrypsin antibody. 125 mM fractions from males with and without prostate cancer were loaded into the wells and incubated for twenty minutes. The sample then was removed and placed in a second well (coated with the same antibody) and incubated for an additional 10 minutes. This process was repeated until the sample had passed through five wells. After the final well, the sample was removed and tested by SELDI for the presence of the 50.76 kD protein. Furthermore, the wells were washed with 50 mM phosphate buffer, pH 7.0, and tested by SELDI for the presence of the 50.76 kD protein. As shown in FIGS. 4A and 4B, the 50.76 kD protein was not retained by the anti-fetuin antibody (protein present in the flow-through (FIG. 4A), but not in the retentate (FIG. 4B)). As shown in FIGS. 4C and 4D, the 50.76 kD protein was not retained by the anti-α-1-antitrypsin antibody (protein present in the flow through (FIG. 4C), but not in the retentate (FIG. 4D)). FIGS. 4E and 4F, however, show that the 50.76 kD protein was retained by the anti-VDBP antibody (protein present in the retentate (FIG. 4F), and present at reduced levels in the flow through (FIG. 4E)). This strongly suggests that the 50.76 kD protein is related to VDBP.

### EXAMPLE 5

VDBP-related Proteins as Markers for Other Cancers

Samples from individuals with a cancer other than prostate cancer (and samples from individuals who do not have the cancer) can be analyzed to detect the presence and status of VDBP-related proteins. As in prostate cancer, Vitamin D metabolism is relevant to other tumor types, such as breast cancers. One or more species of VDBP-related proteins may therefore serve as markers in other cancers. Serum samples are preferably fractionated as in Example 1, and the samples tested (e.g. by mass spectrometry or by Western blot) to determine the presence, concentration, or status of VDBP-related proteins in the fractions. Alternatively, the samples are treated with an antibody to VDBP, and the isolated protein then is analyzed by mass spectrometry or other methods to identify any species indicative of the cancer.

### EXAMPLE 6

Sequencing of Prostate Cancer Marker Proteins

Additional prostate cancer-associated proteins based upon the biochemical and mass spectrometry data provided above may be better characterized as described above and/or by using other techniques. For example, samples of the serum can be fractionated using, for example, column chromatography and/or electrophoresis to produce purified protein samples corresponding to each of the proteins identified in Table 1. The sequences of the isolated proteins can then be determined using conventional peptide sequencing methodologies (see Example 3). It is appreciated that the skilled artisan, in view of the foregoing disclosure, would be able to produce an antibody directed against any prostate cancer-associated protein identified by the methods described herein. Moreover, the skilled artisan, in view of the foregoing disclosure, would be able to produce nucleic acid sequences that encode the fragments described above, as well as nucleic acid sequences complementary thereto. In addition, the skilled artisan using conventional recombinant DNA methodologies, for example, by screening a cDNA library with such a nucleic acid sequence, would be able to isolate full-length nucleic acid sequences encoding target prostate cancer-associated proteins. Such full-length nucleic acid sequences, or fragments thereof, may be used to generate nucleic acid-based detection systems or therapeutics.

### EXAMPLE 7

Antibody-based Assay for Detecting Prostate Cancer in an Individual

Once identified, a prostate cancer-associated protein may be detected in a tissue or body fluid sample using numerous binding assays that are well known to those of ordinary skill in the art. For example, as discussed above, a prostate cancer-associated protein may be detected in either a tissue or body fluid sample using an antibody, for example, a monoclonal antibody, which binds specifically to an epitope disposed upon the prostate cancer-associated protein. In such detection systems, the antibody preferably is labeled with a detectable moiety.

The following assay has been developed for tissue samples; however, it is contemplated that similar assays for testing fluid samples may be developed without undue experimentation. A typical assay may employ a commercial immunodetection kit, for example, the ABC Elite Kit from Vector Laboratories, Inc.

A biopsy sample is removed from the patient under investigation in accordance with the appropriate medical guidelines. The sample is applied to a glass microscope slide and the sample fixed in cold acetone for 10 minutes. Then, the slide is rinsed in distilled water and pretreated with a hydrogen peroxide containing solution (2 mL 30% H₂O₂ and 30 mL cold methanol). The slide is rinsed in a Buffer A, which consists of Tris Buffered Saline (TBS) with 0.1% Tween and 0.1% Brij. A mouse anti-prostate cancer-associated protein monoclonal antibody in Buffer A is added to the slide and the slide is then incubated for one hour at room temperature. The slide is washed with Buffer A, and a secondary antibody (ABC Elite Kit, Vector Labs, Inc) in Buffer A is added to the slide. The slide is then incubated for 15 minutes at 37° C. in a humidity chamber. The slides are washed again with Buffer A, and the ABC reagent (ABC Elite Kit, Vector Labs, Inc.) is then added to the slide for amplification of the signal. The slide is incubated for a further 15 minutes at 37° C. in the humidity chamber.

The slide then is washed in distilled water, and a diaminobenzedine (DAB) substrate added to the slide for 4-5 minutes. The slide is rinsed with distilled water, counterstained with hematoxylin, rinsed with 95% ethanol, rinsed with 100% ethanol, and then rinsed with xylene. A cover slip is then applied to the slide and the result observed by light microscopy.

### Equivalents

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced by reference therein.

### INCORPORATION BY REFERENCE

The entire disclosure of each of the aforementioned patent and scientific documents cited hereinabove is expressly incorporated by reference herein.

What is claimed is:

1. A method of diagnosing prostate cancer in an individual comprising:
detecting in a sample isolated from the individual the presence of a polypeptide comprising an amino acid sequence selected from the group consisting of the sequence of SEQ ID NO:1; the sequence of SEQ ID NO:2; the sequence of SEQ ID NO:3; the sequence of SEQ ID NO:5; the sequence of SEQ ID NO:6; the sequence of SEQ ID NO:7; the sequence of SEQ ID NO:8; the sequence of SEQ ID NO:9; the sequence of SEQ ID NO:10; the sequence of SEQ ID NO:11; and a sequence comprising SEQ ID NO:4 and SEQ ID NO:12, wherein the presence of the polypeptide is indicative of prostate cancer in the individual.

2. The method of claim 1, wherein the polypeptide is present in samples from more than fifty percent of males with prostate cancer and absent in samples from more than fifty percent of males without prostate cancer

3. The method of claim 1, wherein the sample comprises prostate tissue.

4. The method of claim 1, wherein the sample comprises a body fluid.

5. The method of claim 4, wherein the body fluid is selected from the group consisting of blood, serum, plasma, sweat, tears, urine, peritoneal fluid, lymph, semen, seminal fluid, seminal plasma, prostatic secretion, spinal fluid, ascitic fluid, saliva, and sputum.

6. The method of claim 5, wherein the body fluid is serum, plasma, or prostatic secretion.

7. The method of claim 1, further comprising the step of measuring the concentration of the polypeptide in the sample.

8. The method of claim 7, further comprising the step of comparing the concentration of the polypeptide with a threshold value, wherein a concentration of the polypeptide greater than or equal to the threshold value is indicative of the presence of prostate cancer.

9. The method of claim 1, wherein the polypeptide is detected by mass spectrometry.

10. A method of diagnosing prostate cancer in an individual, the method comprising the steps of:
(a) contacting a sample from the individual with a binding moiety that binds specifically to a cancer-associated protein to produce a binding moiety-cancer-associated protein complex, the cancer-associated protein comprising an amino acid sequence selected from the group consisting of the sequence of SEQ ID NO:1; the sequence of SEQ ID NO:2; the sequence of SEQ ID NO:3; the sequence of SEQ ID NO:5; the sequence of SEQ ID NO:6; the sequence of SEQ ID NO:7; the sequence of SEQ ID NO:8; the sequence of SEQ ID NO:9; the sequence of SEQ ID NO:10; the sequence of SEQ ID NO:11, and a sequence comprising SEQ ID NO:4 and SEQ ID NO:12; and
(b) detecting the complex, which if present is indicative of prostate cancer in the individual.

11. The method of claim 10, wherein the binding moiety is an antibody.

12. The method of claim 11, wherein the antibody is a monoclonal antibody.

13. The method of claim 11, wherein the antibody is a polyclonal antibody.

14. The method of claim 11, wherein the antibody is labeled with a detectable moiety.

15. The method of claim 14, wherein the detectable moiety comprises a member selected from the group consisting of a radioactive label, a hapten label, a fluorescent label, a chemiluminescent label, a spin label, a colored label, and an enzymatic label.

16. A composition comprising: an isolated polypeptide that
(a) binds to an anion exchange resin comprising quarternary ammonium groups in the presence of a first solution consisting essentially of 50 mM sodium phosphate, pH 7.0,
(b) elutes from the anion exchange resin in the presence of a second solution consisting essentially of 125 mM sodium chloride in 50 mM sodium phosphate, pH 7.0, after the resin has been washed with a solution consisting essentially of 25 mM sodium chloride in 50 mM sodium phosphate, pH 7.0, a solution consisting essentially of 50 mM sodium chloride in 50 mM sodium phosphate, pH 7.0, and a solution consisting essentially of 75 mM sodium chloride in 50 mM sodium phosphate, pH 7.0, and
(c) comprises an amino acid sequence selected from the group consisting of the sequence of SEQ ID NO:1; the sequence of SEQ ID NO:2; the sequence of SEQ ID NO:3; the sequence of SEQ ID NO:5; the sequence of SEQ ID NO:6; the sequence of SEQ ID NO:7; the sequence of SEQ ID NO:8; the sequence of SEQ ID NO:9; the sequence of SEQ ID NO:10; the sequence of SEQ ID NO:11, and a sequence comprising SEQ ID NO:4 and SEQ ID NO:12.

17. The polypeptide of claim 14, wherein the polypeptide has a mass of about 50.8 kD as measured by MALDI-TOF mass spectrometry using a derivatized chip surface.

18. The polypeptide of claim 14, wherein the polypeptide has an affinity for a derivatized, weak cationic exchange chip surface comprising carboxyl groups or for a derivatized chip surface comprising nickel ions.

19. A composition comprising: an isolated polypeptide that
(a) binds to an anion exchange resin comprising quarternary ammonium groups in the presence of a first solution consisting essentially of 50 mM sodium phosphate, pH 7.0,
(b) elutes from the anion exchange resin in the presence of a second solution consisting essentially of 125 mM sodium chloride in 50 mM sodium phosphate, pH 7.0, after the resin has been washed with a solution consisting essentially of 25 mM sodium chloride in 50 mM sodium phosphate, pH 7.0, a solution consisting essentially of 50 mM sodium chloride in 50 mM sodium phosphate, pH 7.0, and a solution consisting essentially of 75 mM sodium chloride in 50 mM sodium phosphate, pH 7.0, and
(c) binds specifically to an anti-VDBP antibody.

20. A method of diagnosing cancer in an individual comprising:
detecting in a sample isolated from the individual a polypeptide that
(a) binds to an anion exchange resin comprising quarternary ammonium groups in the presence of a first solution consisting essentially of 50 mM sodium phosphate, pH 7.0,
(b) elutes from the anion exchange resin in the presence of a second solution consisting essentially of 125 mM sodium chloride in 50 mM sodium phosphate, pH 7.0, after the resin has been washed with a solution consisting essentially of 25 mM sodium chloride in 50 mM sodium phosphate, pH 7.0, a solution consisting essentially of 50 mM sodium chloride in 50 mM sodium phosphate, pH 7.0, and a solution consisting essentially of 75 mM sodium chloride in 50 mM sodium phosphate, pH 7.0, and
(c) comprises an amino acid sequence selected from the group consisting of the sequence of SEQ ID NO:1; the sequence of SEQ ID NO:2; the sequence of SEQ ID NO:3; the sequence of SEQ ID NO:5; the sequence of SEQ ID NO:6; the sequence of SEQ ID NO:7; the sequence of SEQ ID NO:8; the sequence of SEQ ID NO:9; the sequence of SEQ ID NO:10; the sequence of SEQ ID NO:11, and a sequence comprising SEQ ID NO:4 and SEQ ID NO:12, wherein presence of the polypeptide is indicative of cancer in the individual.

21. The method of claim 20, wherein the cancer is prostate cancer.

22. The method of claim 20, wherein the sample comprises prostate tissue.

23. The method of claim 20, wherein the sample comprises a body fluid.

24. The method of claim 23, wherein the body fluid is selected from the group consisting of blood, serum, plasma, sweat, tears, urine, peritoneal fluid, lymph, semen, seminal fluid, seminal plasma, prostatic secretion, spinal fluid, ascitic fluid, saliva, and sputum.

25. The method of claim 24, wherein the body fluid is serum plasma or prostatic secretion.

26. The method of claim 24, further comprising the step of measuring the concentration of the polypeptide in the sample.

27. The method of claim 26, further comprising the step of comparing the concentration of the polypeptide with a threshold value, wherein a concentration of the polypeptide greater than or equal to the threshold value is indicative of the presence of cancer.

28. The method of claim 24, wherein the polypeptide is detected by mass spectrometry.

29. A method of diagnosing cancer in an individual, the method comprising the steps of:
(a) contacting a sample from the individual with a binding moiety that binds specifically to a cancer-associated protein to produce a binding moiety-cancer-associated protein complex, wherein the binding moiety binds specifically to a polypeptide as in any one of claims 16-19; and
(b) detecting the presence of the complex, which if present is indicative of the presence of cancer in the individual.

30. The method of claim 29, wherein the binding moiety is an antibody.

31. The method of claim 30, wherein the antibody is a monoclonal antibody.

32. The method of claim 30, wherein the antibody is a polyclonal antibody.

33. The method of claim 30, wherein the antibody is labeled with a detectable moiety.

34. The method of claim 33, wherein the detectable moiety comprises a member selected from the group consisting of a radioactive label, a hapten label, a fluorescent label, a chemiluminescent label, a spin label, a colored label, and an enzymatic label.

35. The method of claim 10, wherein the polypeptide is present in samples from more than fifty percent of males with prostate cancer and absent in samples from more than fifty percent of males without prostate cancer.

36. The method of claim 20, wherein the polypeptide is present in samples from more than fifty percent of males with prostate cancer and absent in samples from more than fifty percent of males without prostate cancer.

37. The method of claim 29, wherein the polypeptide is present in samples from more than fifty percent of males with prostate cancer and absent in samples from more than fifty percent of males without prostate cancer.

38. A kit for detecting cancer or for evaluating the efficacy of a therapeutic treatment for cancer, the kit comprising in combination:
a receptacle for receiving a tissue or body fluid sample from a mammal;
a binding moiety which binds specifically to a polypeptide as in any one of claims 16-19; and
a reference sample.

39. The kit of claim 35, wherein the reference sample is indicative of an individual without prostate cancer.

40. An isolated, prostate cancer-associated polypeptide, said polypeptide comprising the characteristics of:
detectable at a higher concentration in serum of a human with prostate cancer than in serum of a human without prostate cancer; and
(i) has a molecular weight of about 21 kD as measured by MALDI-TOF mass spectrometry using a derivatized chip surface, binds to an anion ion exchange resin in the presence of a first solution consisting essentially of 50 mM sodium phosphate, pH 7.0, and elutes from the ion exchange resin in the presence of a second solution consisting essentially of 125 mM sodium chloride in 50 mM to sodium phosphate, pH 7.0;
(ii) has a molecular weight of about 23 kD as measured by MALDI-TOF mass spectrometry using a derivatized chip surface, binds to an anion exchange resin in the presence of a first solution consisting essentially of 50 mM sodium phosphate, pH 7.0, and elutes from the anion ion exchange resin in the presence of a second solution consisting essentially of 175 mM sodium chloride in 50 mM sodium phosphate, pH 7.0,
(iii) has a molecular weight of about 25 kD as measured by MALDI-TOF mass spectrometry using a derivatized chip surface, binds to an anion exchange resin in the presence of a first solution consisting essentially of 50 mM sodium phosphate, pH 7.0, and elutes from the ion exchange resin in the presence of a second solution consisting essentially of 125 mM sodium chloride in 50 mM sodium phosphate, pH 7.0,
(iv) has a molecular weight of about 26 kD as measured by MALDI-TOF mass spectrometry using a derivatized chip surface, binds to an anion exchange resin in the presence of a first solution consisting essentially of 50 mM sodium phosphate, pH 7, and elutes from the ion exchange resin in the presence of a second solution consisting essentially of 100 mM sodium chloride in 50 mM sodium phosphate, pH 7.0,
(v) has a molecular weight of about 51 kD as measured by MALDI-TOF mass spectrometry using a derivatized chip surface, binds to an anion exchange resin in the presence of a first solution consisting essentially of 50 mM sodium phosphate, pH 7.0, elutes from an ion exchange resin in the presence of a second solution consisting essentially of 125 mM sodium chloride in 50 mM sodium phosphate, pH 7.0, and has an affinity for a derivatized chip surface bearing nickel ions,
(vi) has a molecular weight of about 60 kD as measured by MALDI-TOF mass spectrometry using a derivatized chip surface and weakly binds an anion exchange resin in the presence of a solution consisting essentially of 50 mM sodium phosphate, pH 7.0, or
(vii) has a molecular weight of about 125 kD as measured by MALDI-TOF mass spectrometry using a derivatized chip surface, binds to an anion exchange resin in the presence of a first solution consisting essentially of 50 mM sodium phosphate, pH 7.0, and elutes from an ion exchange resin in the presence of a second solution consisting essentially of 125 mM sodium chloride in 50 mM sodium phosphate, pH 7.0.

41. The polypeptide of claim 40, wherein the polypeptide of clause (i), or (iv) is further characterized as having an affinity to an chip surface derivatized to comprise hydrophobic moieties.

42. The polypeptide of claim 40, wherein the polypeptide of clause (iii) is further characterized as having an affinity to a chip surface derivatized to comprise carboxyl moieties.

43. The polypeptide of claim 40, wherein the polypeptide of clause (ii), (vi), or (vii) is further characterized as having an affinity to a chip surface derivatized to comprise nickel ions.

44. The polypeptide of claim 40, wherein the prostate cancer is organ-confined prostate cancer.

45. The polypeptide of claim 40, comprising the additional characteristic of being a non-immunoglobulin protein.

46. The polypeptide of claim 40, comprising the additional characteristic of being a non-albumin protein.

47. The polypeptide of claim 40, further comprising an epitope.

48. A method of diagnosing cancer in an individual comprising detecting in a sample isolated from the individual the presence of the polypeptide of claim 40, which if present is indicative of cancer in the individual.

49. The method of claim 48, wherein the cancer is prostate cancer.

50. The method of claim 48, wherein the sample comprises prostate tissue.

51. The method of claim 48, wherein the sample comprises a body fluid.

52. The method of claim 51, wherein the body fluid is selected from the group consisting of blood, serum, plasma, sweat, tears, urine, peritoneal fluid, lymph, semen, seminal fluid, seminal plasma, prostatic secretion, spinal fluid, ascitic fluid, saliva, and sputum.

53. The method of claim 52, wherein the body fluid is serum plasma, or prostatic secretion.

54. A method of diagnosing cancer in an individual, the method comprising the steps of:
(c) contacting a sample from the individual with a binding moiety that binds specifically to a cancer-associated protein to produce a binding moiety-cancer-associated protein complex, wherein the binding moiety binds specifically to the polypeptide of claim 40; and
(d) detecting the presence of the complex, which if present is indicative of the presence of cancer in the individual.

55. The method of claim 54, wherein the binding moiety is an antibody.

56. The method of claim 55, wherein the antibody is a monoclonal antibody.

57. The method of claim 55, wherein the antibody is a polyclonal antibody.

58. The method of claim 55, wherein the antibody is labeled with a detectable moiety.

59. The method of claim 58, wherein the detectable moiety comprises a member selected from the group consisting of a radioactive label, a hapten label, a fluorescent label, a chemiluminescent label, a spin label, a colored label, and an enzymatic label.

60. An isolated binding moiety that binds specifically the polypeptide of claim 40.

61. The binding moiety of claim 60, wherein the moiety is an antibody, an antigen-binding fragment thereof or a biosynthetic antibody binding site.

62. The binding moiety of claim 60, wherein the binding moiety is a monoclonal antibody.

63. A pharmaceutical composition comprising the binding moiety of claim 60 in a pharmaceutically-acceptable carrier.

64. A method of treating cancer in an individual, the method comprising administering to the individual a therapeutically-effective amount of the composition of claim 63.

65. The method of claim 64, wherein the cancer is prostate cancer.

66. An isolated nucleic acid sequence encoding the protein according to any of claims 16-19 or claim 40, or a sequence complementary thereto.

67. An isolated nucleic acid sequence comprising at least 15 nucleotides and capable of hybridizing under stringent hybridization conditions to the nucleic acid of claim 66.

68. An expression vector comprising the nucleic acid of claim 67.

69. A composition comprising the nucleic acid of claim 67 admixed with a pharmaceutically acceptable carrier.

70. A composition comprising the nucleic acid of claim 68 admixed with a pharmaceutically acceptable carrier.

71. A method of treating cancer in an individual, the method comprising introducing into cells of the individual the expression vector of claim 68.

72. The method of claim 71, wherein the cancer is prostate cancer.

73. A method of detecting the presence of prostate cancer in a human, the method comprising detecting the presence of a nucleic acid molecule in a tissue or body fluid sample of the human thereby to indicate the presence of prostate cancer in the human, wherein the nucleic acid molecule comprises a nucleic acid sequence encoding at least a portion of a prostate cancer-associated protein according to any of claims 16-19 or claim 40, or a nucleic acid sequence capable of recognizing and being specifically bound by the prostate cancer-associated protein.

74. The method of claim 73, wherein the method comprises the step of reacting the sample with a labeled hybridization probe capable of hybridizing specifically to the nucleic acid molecule.

75. A method of detecting the presence of cancer in an individual, the method comprising the steps of:
(a) exposing a sample from the individual under specific hybridization conditions to a nucleic acid probe capable of hybridizing specifically to a target nucleic acid encoding a polypeptide according to any of claims 16-19 or claim 40; and
(b) detecting the presence of a duplex comprising the nucleic acid probe, the presence of the duplex being indicative of cancer in the individual.

76. The method of claim 75 further comprising the step of amplifying the target nucleic acid in the sample prior to exposing the sample to the nucleic acid probe.

77. The method of claim 75, wherein the cancer is prostate cancer.

78. The method of claim 75, wherein the nucleic acid probe is labeled with a detectable moiety.

79. The method of claim 78, wherein the detectable moiety comprises a member selected from the group consisting of a radioactive label, a hapten label, a fluorescent label, and an enzymatic label.

80. A kit for detecting the presence of prostate cancer or for evaluating the efficacy of a therapeutic treatment of a prostate cancer, the kit comprising in combination:
a receptacle for receiving a tissue or body fluid sample from a mammal;
a binding moiety which binds specifically to the prostate cancer-associated protein of claim 40; and
a reference sample.

81. The kit of claim 80, wherein the reference sample is indicative of an individual without prostate cancer.


SEQUENCE LISTING (14 sequences)

SEQ ID NO: 1 - protein, 14 aa, Homo sapiens
FPSGTFEQVSQLVK

SEQ ID NO: 2 - protein, 15 aa, Homo sapiens
KFPSGTFEQVSQLVK

SEQ ID NO: 3 - protein, 7 aa, Homo sapiens
TSALSAK

SEQ ID NO: 4 - protein, 5 aa, Homo sapiens
EGLER

SEQ ID NO: 5 - protein, 11 aa, Homo sapiens
HLSLLTTLSNR

SEQ ID NO: 6 - protein, 8 aa, Homo sapiens
LCDNLSTK

SEQ ID NO: 7 - protein, 7 aa, Homo sapiens
YTFELSR

SEQ ID NO: 8 - protein, 11 aa, Homo sapiens
VMDKYTFELSR

SEQ ID NO: 9 - protein, 10 aa, Homo sapiens
THLPEVFLSK

SEQ ID NO: 10 - protein, 11 aa, Homo sapiens
RTHLPEVFLSK

SEQ ID NO: 11 - protein, 7 aa, Homo sapiens
VLEPTLK

SEQ ID NO: 12 - protein, 5 aa, Homo sapiens
GPLLK

SEQ ID NO: 13 - DNA, 1652 bp, Homo sapiens
  CDS (28)..(1452)
ggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccat

SEQ ID NO: 14 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL